(19) United States
(12) Patent Application Publication
(10) Pub. No.: US 2025/0263797 A1
(43) Pub. Date: Aug. 21, 2025

(54) CLEAR CELL RENAL CELL CARCINOMA BIOMARKERS AND USES THEREFOR

(71) Applicants: VILNIUS UNIVERSITY, Vilnius (LT); THERMOPHARMA BALTIC, UAB, Vilnius (LT)

(72) Inventors: Raimonda KUBILIUTE, Vilnius (LT); Sonata JARMALAITE, Vilnius (LT)

(21) Appl. No.: 18/268,801

(22) PCT Filed: Mar. 26, 2021
(86) PCT No.: PCT/IB2021/052532

§ 371 Date: Jun. 21, 2023

Related U.S. Application Data
(60) Provisional application No. 63/128,874, filed on Dec. 22, 2020.

(51) Int. Cl. C12Q 1/6886

(52) U.S. Cl. CPC C12Q 1/6886; C12Q 2600/118; C12Q 2600/154

(57) ABSTRACT

The present invention refer to biomarkers, methods and tools for the detection, diagnosis, prognosis and monitoring/active surveillance of the individuals having clear cell renal cell carcinoma or small renal masses. The methods consist of obtainment of biological sample (tissues or urine) and determination of the DNA methylation status and/or methylation level of at least one of biomarkers, including ZNF677, FBN2, PCDH8, TFAP2B, TAC1, FLRT2, SFRP1, ADAMTS19, BMP7 and SIM1, Moreover the described invention provides the kits, primers and probes for use in such a method.

### FIELD OF THE INVENTION

The present application related generally to biomarkers and methods for identifying clear cell renal cell carcinoma (ccRCC), diagnosing ccRCC and predicting ccRCC progression whereby DNA methylation status and/or level of particular epigenetic biomarkers or a combination thereof are detected and measured in vitro in renal tissues and/or body fluids, such as urine. The invention also provides the kits and oligonucleotides for performing the assays.

### BACKGROUND OF THE INVENTION

Kidney and renal pelvis cancer take 14th place according to incidence in both sexes worldwide and is among the top ten most common cancers in males [according to 2018 data from Cancer Today, Global Cancer Observatory, International Agency for Research on Cancer; https://gco.iarc.fr/today/home]. Most frequent type of renal neoplasm is renal cell carcinoma (RCC), which accounts for 85-90% of all renal malignancies and is the most lethal cancer of the urinary system [1,2]. Meanwhile remaining ˜10% of kidney cancers composed of urothelial carcinoma of renal pelvis [1]. RCC encompasses a heterogeneous group of cancers derived from renal tubular epithelial cells with the major subtypes of clear cell (also named as conventional) RCC (ccRCC; 70-80%), papillary RCC (pRCC; 10-15%) and chromophobe RCC (chRCC; 5%). The remaining subtypes are very rare (each with≤1% total incidence) [3]. ccRCC is the most common subtype which arises from the proximal convoluted tubule and is characterized with the predominance of metastatic disease (˜90%) as well as accounts for the majority of deaths from kidney cancer [3] and is the focus of this invention. Besides RCC type, histological diagnosis includes evaluation of nuclear grade, sarcomatoid features, vascular invasion, tumour necrosis, and invasion of the collecting system and peri-renal fat, pT, or even pN categories.

RCC is staged by the size of the tumour and its presence in the other tissues, including renal vein, perirenal or renal sinus fat, renal fascia and lymph nodes. In stage pT1, the cancer is considered less or equal to 7 cm and limited to the kidney without spreading into lymph nodes (N0) or distant organs (M0). In more specifically, pT1a stage tumours is considered as clinically localized renal masses defined as ≤4 cm in diameter while pT1b stage tumours is larger than 4 cm. In stage pT2, the tumours is greater than 7 cm (pT2a≤10 cm and pT2b>10 cm in the detail), but still limited to the kidney. In stage pT3, cancer cells are found in mojour veins (vena cava) or perinefric tissues but not into the adrenal gland and not beyond Gerota fascia (connective tissue surrounding the kidneys and the adrenal glands). The cancer may spread to nearby lymph nodes (N1) but has not spread to other organs (M0). In the detail, pT3a stage tumours grossly extends into the renal vein or its segmental (muscle-containing) branches, or tumour invades perirenal and/or renal sinus fat (peripelvic fat), but not beyond Gerota fascia; T3b tumours grossly extends into the vena cava below diaphragm; T3c tumours grossly extends into vena cava above the diaphragm or invades the wall of the vena cava. T4 tumours invade beyond Gerota fascia and may be growing into the adrenal gland. It may or may not have spread to nearby lymph nodes (any N) and distant lymph nodes and/or other organs (M0 or 1).

Without tumour stage, tumour grade and necrosis contribute to ccRCC prognosis as well. Fuhrman nuclear grade is the most widely accepted grading system which concurrently evaluates nuclear size and shape, and nucleolar prominence. Fuhrman nuclear grade is an independent prognostic factor [EAU guidelines on Renal Cell Caecinoma; limited update 2020], however due to intra- and inter-observer variability, becomes inapplicable. The new WHO/ISUP (International Society of Urologic Pathologists) grading system replaced the Fuhrman grading and provided superior prognostic information in cases of ccRCC [4]. Studies suggest that adding the presence of tumour-associated necrosis into the ISUP/WHO grading system improves outcome predictions as it is an independent prognostic factor for ccRCC [5].

Clinically renal cell carcinoma is associated with few symptoms; i.e. flank pain, hematuria or a palpable abdominal mass, but these signs are rare (6-10%) and correlates with aggressive histology and advanced disease [6]. But most of cases are asymptomatic and nowadays mostly discovered incidentally due to extensive use of abdominal computed tomography (CT), ultrasound (US), and magnetic resonance tomography (MRT) for other medical reasons [7, 8, 9]. The sensitivity of US is extremely low in the case of renal masses smaller than 1 cm (26%) [10]. Although sensitivity and specificity of CT and MRT reach 100% and ≥90% respectively, none of these methods are able to distinguish benign renal lesions from neoplastic [11, 12]. Among patients with local disease, 5-years survival rate reach 95% [13], but still 20-30% of patients with local disease at diagnosis will develop metastasis after nephrectomy [14]. If metastases are present at diagnosis, the prognosis are much worse ant the probability of 5-year survival may be as low as 10-15% [14]. Regarding to this, in addition to conventional imagining procedures, TNM staging and histological grading, there is a need for new molecular biomarkers for early diagnosis of ccRCC or to identify patients with high risk of progress.

For patients with surgically resectable RCC, the standard of care is surgical excision by either partial or radical nephrectomy with a curative intent [13]. Biopsy was discouraged due to fear of spread of tumour cells along the biopsy tract. As such, some patients were over treated for benign or indolent renal tumours, (pT1a stage and ≤4 cm in diameter) also known as small renal masses (SRM) who are incidentally detected via abdominal imaging. Active surveillance is an oncologically safe option in slow growing SRM, defined as initial monitoring of tumour size by serial abdominal US with delayed treatment [15, 28]. In order to low sensitivity of US for small renal tumours [10], biomarkers that could not only to detect even small indolent renal lesions, but also be able to lead the way for treatment decisions during active surveillance of the patients diagnosed with SMR, are needed.

DNA methylation in mammalian cells is characterized by the addition of a methyl group (—CH3) at the carbon-5 position of cytosine residues in the context of CpG dinucleotides through the action of DNA methyltransferase enzymes, forming 5-methylcytosine (5mC). It is the most widely studied epigenetic mechanizm, responsible for the various biological processes including normal development of the mammals, differentiation and regulation of gene expression [16]. There are approximately 28 million CpG sites in the genome, but these are not evenly distributed; a part of CpG dinucleotides in the genome of mammals tends to concentrate into short CpG-rich regions, called CpG island, which are located in the promoters of the genes (around the transcription start sites) or other regulatory sequences [17]. Promoter CpG islands in normal cells generally remain unmethylated (hypomethylated) and are associated with active gene expression during differentiation. On the contrary, methylated CpG island promoters (hypermethylation) usually are associated with gene repression [16].

Normal epigenetic processes, including genome-wide changes in DNA methylation pattern, are disrupted during the initiation and progression of cancer [18, 19]. Hypermethylation of the CpG islands is a common event in various cancer types, including kidney cancer and is often associated with the silencing of tumour suppressor genes and downstream signaling pathways [16, 20]. During renal cell carcinoma carcinogenesis, mutation in genes, which are responsible for epigenetic regulation, are observed and lead abundant changes in DNA methylation, compared to normal kidney samples [21]. In the comparison with genetic alterations, DNA hypermethylation is more pronounced and frequently found in all RCC subtypes, including ccRCC. Because DNA methylation is considered as common and one of the earliest observable molecular changes in cancer, that is easily detectable in small amounts of DNA, these alterations could be interesting clinical cancer biomarkers for disease diagnosis, prognosis, and directed therapies [22-24]. However, despite of their potential, no diagnostic or prognostic RCC DNA methylation biomarker has reached the clinic yet.

Regarding the implementation possibilities in clinical practice and other above mentioned advantages, DNA methylation have several additional superiority. Compared to RNA transcripts or protein-based biomarkers, DNA methylation is much more stable, besides can be amplified and thus increase sensitivity, which allows detection of such biomarkers on limited amounts of samples. Urological cancers, especially in case or renal cancer, derived methylated DNA can be easily detectable in the urine samples, which allows the development of non-invasive molecular tests [25]. Furthermore, as ccRCC is considered to be heterogenous malignancy with high intra-tumour and inter-tumour heterogeneity [21, 26, 27], which complicates identification of novel cancer biomarkers, however DNA methylation in urine samples bypasses this situation because of better reflection of tumour heterogeneity compared to tissue sample. Moreover, urine-based biomarkers can be checked frequently which is especially important for renal cancer, where biopsy in most cases are not available. In addition, such an easily available repeatability of sample acquisition, allows to detect cancer at an early stage or to follow the real-time state of the malignant transformation.

This invention presents a set of DNA methylation biomarkers, characterized by the potential clinical benefits and provides a potential non-invasive tool for the early detection of clear cell renal carcinoma.

### SUMMARY OF THE INVENTION

The present application includes biomarkers, methods, reagents, devices, systems, and kits for the detection and diagnosis of renal cancer and more particularly, clear cell renal cell carcinoma (ccRCC) subtype. More specifically, we have found that alterations of DNA methylation status and/or DNA methylation level of a set of genomic loci including the genes ZNF677, TFAP2B, TAC1, FLRT2, ADAMTS19, BMP7 and SIM1 are associated with ccRCC and can be used as biomarkers for ccRCC detection, diagnostics, prognosis and patients' monitoring when analysed individually or in various combinations. Additionally, previously analysed biomarkers namely FBN2, PCDH8 and SFRP1 can be included in the biomarker panel providing increased diagnostic and/or prognostic value of the test.

According to one aspect of the invention a panel of DNA methylation biomarkers consisting of ZNF677 (SEQ ID NO: 1), TFAP2B (SEQ ID NO: 4), TAC1 (SEQ ID NO: 5), FLRT2 (SEQ ID NO: 6), ADAMTS19 (SEQ ID NO: 8), BMP7 (SEQ ID NO: 9) and SIM1 (SEQ ID NO: 10) is provided for identification and/or characterization of ccRCC, and/or prognosis of the course of disease in a test samples containing nucleic acids from renal tissues, urine or cells obtained from an patients diagnosed with ccRCC. Additionally, FBN2 (SEQ ID NO: 2), PCDH8 (SEQ ID NO: 3) and SFRP1 (SEQ ID NO: 7) and can be included in the panel.

According to another aspect, a method, based on qualitative methylation-specific PCR (MSP), is provided for detection of at least one of the DNA methylation biomarkers from the panel consisting of ZNF677 (SEQ ID NO: 1), TFAP2B (SEQ ID NO: 4), TAC1 (SEQ ID NO: 5), FLRT2 (SEQ ID NO: 6), ADAMTS19 (SEQ ID NO: 8), BMP7 (SEQ ID NO: 9), SIM1 (SEQ ID NO: 10) as well as FBN2 (SEQ ID NO: 2), PCDH8 (SEQ ID NO: 3) and SFRP1 (SEQ ID NO: 7) in a investigated sample containing nucleic acids from renal tissues, urine or cells obtained from an individuals diagnosed with ccRCC.

In another aspect, the invention provides a second panel of biomarkers consisting of ZNF677 (SEQ ID NO: 11), TFAP2B (SEQ ID NO: 14) TAC1 (SEQ ID NO: 15) and FLRT2 (SEQ ID NO: 16) in addition to FBN2 (SEQ ID NO: 12) and PCDH8 (SEQ ID NO: 13) for identification or diagnosis of ccRCC, characterization of ccRCC, prognosis of ccRCC and monitoring of individuals diagnosed with ccRCC, in a test sample containing nucleic acids from renal tissues, urine or cells obtained from an individuals.

In another aspect, the described invention provides a second method, based on quantitative methylation-specific PCR (QMSP), for detection of at least one of the DNA methylation biomarkers from the panel consisting of ZNF677 (SEQ ID NO: 11), TFAP2B (SEQ ID NO: 14) TAC1 (SEQ ID NO: 15) and FLRT2 (SEQ ID NO: 16) as well as FBN2 (SEQ ID NO: 12) and PCDH8 (SEQ ID NO: 13) in a test sample containing nucleic acids from urine obtained from an individuals.

In another embodiment of the invention, a kits for estimating qualitatively or/and quantitatively at least one of the methylation biomarkers from the panel consisting of ZNF677 (SEQ ID NO: 1 or/and SEQ ID NO: 11), TFAP2B (SEQ ID NO: 4 or/and SEQ ID NO: 14), TAC1 (SEQ ID NO: 5 or/and SEQ ID NO: 15), FLRT2 (SEQ ID NO: 6 or/and SEQ ID NO: 16), ADAMTS19 (SEQ ID NO: 8), BMP7 (SEQ ID NO: 9) and SIM1 (SEQ ID NO: 10) as well as FBN2 (SEQ ID NO: 2 or/and SEQ ID NO: 12), PCDH8 (SEQ ID NO: 3 or/and SEQ ID NO: 13) and SFRP1 (SEQ ID NO: 7) in a test sample containing nucleic acids from renal tissues, urine or cells obtained from an individual.

An additional aspect of the invention provides primers and probes for the detection of the methylation biomarkers in a test sample of any kind of human-derived tissue, cells, body fluid or nucleic acids obtained from human-derived tissue, cells or body fluid. A particular primer or probe comprises a nucleotide sequence selected from the group consisting of SEQ ID NOS: 18-75.

### BRIEF DESCRIPTION OF THE FIGURES

The invention is illustrated with the following figures.

FIG. 1. Venn diagrams of the genes with significantly different methylation levels according to renal tissue histology and tumour stages. The lists of differentially methylated genes were obtained by means of DNA methylation microarrays. ccRCC—clear cell renal cell carcinoma; NRT—noncancerous renal tissue.

FIG. 2. Gene set enrichment analysis (GSEA) of hypermethylated and hypomethylated genes identified in the genome-wide methylation profiling. Only genes having significant methylation differences with fold change values ≥1.5 are included. The collection of Hallmark pathways as defined in MSigDB were selected for the enrichment analysis. The gray scale indicates the false discovery rate (FDR)-adjusted P-value (q-value). ccRCC—clear cell renal cell carcinoma; NRT—noncancerous renal tissue.

FIG. 3. Volcano plots of DNA methylation profiling in renal tissues. A—methylation differences between ccRCC and NRT; B—methylation differences in pTa stage tumours compared to NRT; C—methylation differences in pT4 stage tumours compared to NRT. Gray-shaded squares indicate probes, coloured according to the fold change values (FC≥1.5) and P-values (<0.0500). Labels indicate microarray probes of the genes selected for further validation analysis.

FIG. 4. Methylation frequencies of the genes ZNF677, FBN2, PCDH8, TFAP2B, TAC1, FLRT2, SFRP1, ADAMTS19, BMP7 and SIM1 in renal tissues. The results were obtained by qualitative methylation-specific PCR (MSP). ccRCC—clear cell renal cell carcinoma, NRT—noncancerous renal tissues. Significant P-values are in bold.

FIG. 5. Methylation frequencies of the genes ZNF677, FBN2, PCDH8, TFAP2B, TAC1, FLRT2, SFRP1, ADAMTS19, BMP7 and SIM1 in renal tumour tissues according to the demographica variables of the investigated cohort, including patients' gender (A) and age at diagnosis of clear cell renal cell carcinoma (B). The box extends from the 25th to 75th percentiles; the line in the box is plotted at median; the plus sign depicts the mean; the whiskers represent the 10-90% range; data values outside the range are marked as dots. M—methylated, U—unmethylated gene status. Significant P-values are in bold.

FIG. 6. Methylation frequencies of the genes ZNF677, FBN2, PCDH8, TFAP2B, TAC1, FLRT2, SFRP1, ADAMTS19, BMP7 and SIM1 in renal tumour tissues according to the clinical-pathological variables, including tumour stage (A), tumour size (B), Fuhrman grade (C), differentiation grade (D), intravascular invasion (E) and tumour necrosis (F). The box extends from the 25th to 75th percentiles; the line in the box is plotted at median; the plus sign depicts the mean; the whiskers represent the 10-90% range; data values outside the range are marked as dots. M—methylated, U—unmethylated gene status. Significant P-values are in bold.

FIG. 7. Kaplan-Meier curve analysis of patients' gender (A), patients's age (B), tumour stage (C), tumour size (D), tumour differentiation grade (E), tumour intravascular invasion (F), tumour fat invasion (G), tumour necrosis (H), methylation status of ZNF677 (I), FBN2 (J) and combination of two-four genes (K—O). M—methylated and U—unmethylated gene status. Significant P-values are in bold.

FIG. 8. Relative expression levels of the genes ZNF677 (A), FBN2 (B), PCDH8 (C), TFAP2B (D), TAC1 (E) and FLRT2 (F) in the renal tissues. The box extends from the 25th to 75th percentiles; the line in the box is plotted at median; the plus sign depicts the mean; the whiskers represent the 10-90% range. ccRCC—clear cell renal cell carcinoma, NRT—noncancerous renal tissues. Significant P-values are in bold.

FIG. 9. Relative expression of the genes ZNF677 (A), FBN2 (B), PCDH8 (C), TFAP2B (D), TAC1 (E) and FLRT2 (F) in renal tissues according to the methylated (M) or unmethylated (U) gene status. The box extends from the 25th to 75th percentiles; the line in the box is plotted at median; the plus sign depicts the mean; the whiskers represent the 10-90% range. Significant P-values are in bold.

FIG. 10. Methylation levels of ZNF677, FBN2, PCDH8, TFAP2B, TAC1 and FLRT2 in urine of patients diagnosed with clear cell renal cell carcinoma (ccRCC) and asymptomatic (healthy) control (ASC) cases. The results were obtained by quantitative methylation-specific PCR (QMSP). Whiskers represent the standard error of mean. Significant P-values are in bold.

FIG. 11. Receiver Operating Characteristic (ROC) curve analysis of the methylation biomarkers, as diagnostics of renal cancer, in urine of the patients diagnosed with clear cell renal cell carcinoma (ccRCC). A-ZNF677, B-FBN2, C-PCDH8, D-TFAP2B, E-TAC1, and F—I—combination of two-five genes. AUC—area under the curve. Significant P-values are in bold.

FIG. 12. Methylation frequencies of ZNF677, FBN2, PCDH8, TFAP2B, TAC1 and FLRT2 in urine of patients diagnosed with clear cell renal cell carcinoma (ccRCC) and asymptomatic (healthy) control (ASC) cases. Significant P-values are in bold.

FIG. 13. Correlations between promoter methylation level and patients' age at the diagnosis of renal cancer for ZNF677 (A), FBN2 (B), PCDH8 (C), TFAP2B (D), TAC1 (E) and FLRT2 (F). Spearman's R (Rs) correlation coefficients are provided with respective P-values. Significant P-values are in bold.

FIG. 14. The association of renal tumour size with methylation status of ZNF677, FBN2, PCDH8, TFAP2B, TAC1 and FLRT2 (A) in urine saples of patients diagnosed with clear cell renal cell carcinoma (ccRCC) and methylation levels of the same genes according to tumour fat invasion (B). The box extends from the 25th to 75th percentiles; the line in the box is plotted at median; the plus sign depicts the mean; the whiskers represent the 10-90% range. M—methylated, U—unmethylated gene status. Significant P-values are in bold.

FIG. 15. Methylation levels (A) and frequencies (B) of ZNF677, FBN2, PCDH8, TFAP2B, TAC1 and FLRT2 in urine of patients diagnosed with small renal masses (SRM) and asymptomatic (healthy) control (ASC) cases. The results were obtained by quantitative methylation-specific PCR (QMSP). Whiskers represent the standard error of mean. Significant P-values are in bold.

FIG. 16. Methylation levels (A) and frequencies (B) of ZNF677, FBN2, PCDH8, TFAP2B, TAC1 and FLRT2 in urine of patients' diagnosed with small renal masses (SRM) at different time points (0, 6, 12, 18, 24 months) of the follow-up. Whiskers represent the standard error of mean. Significant P-values are in bold.

### DETAILED DESCRIPTION OF THE INVENTION

The present invention provides the set of biomarkers, the methods and the kits useful for diagnosing ccRCC, predicting ccRCC progression and performing patient's monitoring (active surveillance). While the invention will be described in conjunction with the following terminology and enumerated embodiments, it is not intended to limit the field of application of the invention to those embodiments. The present invention is in no way restricted to the methods and materials described in the Materials and Methods section herein, as these may involve all modifications and alternatives that may be included within the scope of described invention as defines by the claims.

### Definitions

Unless defined otherwise, all other scientific and technical terms have the meaning as commonly understood by those of ordinary skill in the art.

As used in this application, including the claims, the singular forms “a,” “an,” and “the include plural references as well, unless the content clearly dictates otherwise.

As used herein, “renal” may be interchangeably referred to as “kidney”.

The terms “biomarker”, “epigenetic biomarker”, “DNA methylation biomarker” or “methylation biomarker” are used interchangeably in this invention and refers to a genomic loci that is aberrantly methylated, where the DNA methylation status or/and the DNA methylation level indicate the presence or the absence of clear cell renal cell carcinoma or/and small renal masses.

As used herein, the term “primer” represents a nucleic acid of at least 18 nucleotides in length which is made synthetically and in the presence of certain conditions can hybridize according complementarity to any of the biomarker sequences from the group of SEQ ID NOs: 1-17. The primer can act as a start site of synthesis of a complementary DNA strand.

The term “probe” as used herein represents a primer labelled with one or two tags which are detectable by measuring fluorescence and with one quencher molecules or the like, i.e. TaqMan® probes. In a present invention the probes from the group of SEQ ID NO: 60, SEQ ID NO: 63, SEQ ID NO: 66, SEQ ID NO: 69, SEQ ID NO: 72, SEQ ID NO: 75 and SEQ ID NO: 78 are labelled with FAM at the 5′ and with BHQ-1 at the 3′ end.

One skilled in the art will recognize the method of “bisulfite conversion”, comprising the step of treating DNA with bisulfite and thereby converting non-modified (non-methylated) cytosine to uracil, whereas methylated cytosine remains remains unchanged. In addition, the purification step of the converted DNA can be included in the protocol. All steps of this method can be performed manually, or by using a commercially available kits.

The terms “methylation” and “DNA methylation” are used interchangeably in this embodiment and in the claims and refer to methylation at the carbon-5 position of cytosine. “Unmethylated DNA” or “methylated DNA” indicate to the naturally (wild-type) unmethylated or methylated DNA loci or to amplified DNA sequence after bisulfite conversion which was originally unmethylated or methylated.

The terms “DNA methylation status” and “methylation status are used herein interchangeably and are intended to cover the presence or absence of methylation according to the particular biomarker. The presence of DNA methylation can also be named as “DNA hypermethylation” or “hypermethylation”.

The terms “DNA methylation level” or “methylation level” are interchangeable and refers to the quantity of methylation according to one or more of the biomarkers. The methylation level according to a particular biomarker can be expressed as a relative or absolute value, additionally but not necessarily normalized to a standard or a reference sample (or samples). The value can also be expressed as a percentage or a proportion of a standard sample or a reference sample.

The term “threshold” refer to means a specific methylation level above which the results are considered as positive or having a positive methylation status, whereas otherwise the results are classified as negative or having a negative methylation status.

The term “sample” and “test sample” are used interchangeably herein to refer to any material, biological fluids, tissue, or cell obtained or otherwise derived from an individual. This includes urine, cells, tissues preferably from an individual suspected of having renal cancer, or nucleic acids from tissues, cells or urine. The sample can be obtained from a patient diagnosed with renal cancer or healthy individual or an individual with the unknown state of health.

The terms “individual” and “patient” are used interchangeably herein and indicate a human both male, o female.

The term “progression”, as generally understood in the field of oncology, indicate the unfavorable changes in characteristics of the disease including clinical-pathological parameters, symptoms of the disease, new cancerous lesions (metastases), patient's death and so on.

The terms “diagnosing”, “diagnosis’, and variations thereof refer to the detection, determination, or recognition of a health status or condition of an individual on the basis of one or more signs, symptoms, data, or other information assign to that individual. The terms “diagnosing” “diagnosis” and so on cover, the initial detection of the disease; the characterization or classification of the disease; the detection of the progression of the disease and the detection of disease response after the treatment of the individual. The diagnosis of ccRCC includes distinguishing individuals who have cancer from individuals who do not.

The term “prognosis” refer to the prediction of a future course of a disease or condition in an individual who has the disease or condition (e.g., predicting patient survival), and such term involve the assessment of disease response after the administration of a treatment to the individual.

As used in this application, the term “kit” refer to a set of reagents and/or tools and/or equipment optionally including instructions (protocols) for the use of the mentioned set.

### DNA Methylation Biomarkers and Detection Thereof

The present invention encompass genomic loci that are susceptible for the alterations in DNA methylation in the context of renal carcinogenesis and tumour (specially clear cell renal cell carcinoma) development. Cytosines within CpG dinucleotides in the particular genomic loci analysed in test samples are differentially methylated in ccRCC tissues and noncancerous renal tissues (NRT). Precisely, the methylation of the genomic loci is more frequent and/or at a higher level in tumours and less common and/or at a lower level in NRT or asymptomatic (healthy) individuals. The differences of methylation were found in the genomic sequences, which are covered by known genes with publicly available descriptions in specialized databases, e.g., GeneBank® of the National Institutes of Health (USA). In certain embodiments, the biomarkers include one or more of ZNF677 (SEQ ID NO: 1 or/and SEQ ID NO: 11), TFAP2B (SEQ ID NO: 4 or/and SEQ ID NO: 14), TAC1 (SEQ ID NO: 5 or/and SEQ ID NO: 15), FLRT2 (SEQ ID NO: 6 or/and SEQ ID NO: 16), ADAMTS19 (SEQ ID NO: 8). BMP7 (SEQ ID NO: 9) and SIM1 (SEQ ID NO: 10) including FBN2 (SEQ ID NO: 2 or/and SEQ ID NO: 12), PCDH8 (SEQ ID NO: 3 or/and SEQ ID NO: 13) and SFRP1 (SEQ ID NO: 7). The DNA methylation biomarkers in the present invention are DNA sequences that contain CpG dinucleotides and are prone to differential methylation. The antisense sequence of the genetic locus containing a biomarker can be utilized. The said antisense biomarker sequence can be analysed with the primers designed easily by a person skilled in the art.

In a preferred embodiment, methods for DNA amplification (e.g. polymerase chain reaction, PCR) can be used to quantify DNA within a locus surrounded by primers. At first, genomic DNA is treated with bisulfite in order to convert unmodified (unmethylted) cytosines to uracils, whereas methylated cytosines remain unchanged, that allow to create an unnatural sequence illustrating cytosine modification status in the native DNA. All steps of bisulfite conversion can be performed manually, or by using a commercially available kits or both. Then the amplification of the target DNA sequence is performed by primers that complementary hybridize to sequence of bioarker. In one embodiment, for qualitative evaluation of DNA methylation two primer pairs, specific to methylated and corresponding unmethylated sequence, are sed to amplify the bisulfite-converted DNA. The presence of amplification products with primer pairs, specific to methylated/unmethylated DNA sequence indicates the methylated/unmethylated status of the investigated loci. In another embodiment, for quantitative evaluation of DNA methylation, primers and probes specific only for methylated DNA can be used for the amplification.

In the preferred embodiment, DNA methylation status of a biomarker panel consisting of ZNF677 (SEQ ID NO: 1), TFAP2B (SEQ ID NO: 4), TAC1 (SEQ ID NO: 5), FLRT2 (SEQ ID NO: 6), ADAMTS19 (SEQ ID NO: 8), BMP7 (SEQ ID NO: 9), SIM1 (SEQ ID NO: 10) as well as FBN2 (SEQ ID NO: 2), PCDH8 (SEQ ID NO: 3) and SFRP1 (SEQ ID NO: 7) is analysed by qualitative methylation specific PCR (MSP) using a primers for the methylated and unmethylated sequence indicated by SEQ ID NOs: 18-57. According to the presence and/or absence of the specific amplification products with the corresponding primer pairs, the DNA methylation status of each biomarker analysed is evaluated as methylated or unmethylated. The in vitro methylated and unmethylated controls are included in the assay to eliminate the technical interference of the method.

In the preferred embodiment, DNA methylation levels of a biomarker from the panel consisting of ZNF677, FBN2, PCDH8, TFAP2B, TAC1 and FLRT2 indicated by SEQ ID NOs: 11-16 are analysed by means of quantitative methylation-specific PCR (QMSP) using a set of primer pair and a probe specific for the methylated sequence. Alternatively, at least one of the component in a set of primer pair and probe may be specific for the methylated sequence of biomarkers. An endogenous control gene, e.g. ACTB (SEQ ID NO: 17), is analysed simultaneously, to normalize the sample input. Preferably, the TaqMan hydrolysis probes labelled with FAM or other tag at 5′-end and a quencher moiety at 3′-end, e.g. BHQ1 are used for the biomarker assays. The in vitro methylated control is preferably included as the standard of methylated DNA and used as a reference sample to estimate the methylation level of a biomarker.

In one embodiment, the methylation level of the biomarker panel consisting of ZNF677, FBN2, PCDH8, TFAP2B, TAC1 and FLRT2 indicated by SEQ ID NOs: 11-16 is evaluated by the cycle of quantification (Cq) value obtained by determination of fluorescence signal intensity at a particular cycle of the QMSP reaction. The DNA methylation level of a particular biomarker in a sample can be provided as a percentage of the DNA methylation level in the methylated control.

In another embodiment, the methylation level of the biomarker panel consisting of ZNF677, FBN2, PCDH8, TFAP2B, TAC1 and FLRT2 indicated by SEQ ID NOs: 11-16 can be established from the DNA methylation level. The particular biomarker in the test sample can be considered as methylated if the evaluated methylation level is more than calculated threshold value. The biomarker in the test sample is considered as unmethylated if their methylation level is less or equal to the threshold value. In the preferred embodiment, the threshold value is selected based on the average DNA methylation level of the particular biomarker in a samples, obtained from asymptomatic (healthy) individuals and is different for each biomarker.

Kits for the Detection of ccRCC Biomarkers

The present invention encompass the kits for the detection and evaluation the DNA methylation status and/or methylation level of methylation biomarkers, described herein. The kits consist of primers and/or probs, that complementary hybridizes to at least one of the biomarker sequence, and other reagents/components for detecting biomarker methylation.

In some embodiments, the kits consist of one or more of nucleotide sequence that hybridize and amplify any part of a genomic sequence of described biomarkers, precisely ZNF677, FBN2, PCDH8, TFAP2B, TAC1, FLRT2, SFRP1, ADAMTS19, BMP7 and SIM1 specified by SEQ ID NOs: 1-16.

In some embodiments, the kits comprise at least one of the primers or probes specified by SEQ ID NOs: 18-75 specifically hybridizing to any fragment of the biomarkers, including ZNF677, FBN2, PCDH8, TFAP2B, TAC1, FLRT2, SFRP1, ADAMTS19, BMP7 and SIM1. In some embodiments, the kits can also involve sodium bisulfite together with at least one of the primers/probes specified by SEQ ID NOs: 18-75.

Biomarkers for ccRCC Detection and Diagnosis

The invention describe the diagnostic tools to identify ccRCC. The biomarkers described herein are differentially methylated in cancerous and histologically unchanged (normal/healthy) renal tissue samples and for this reason are advantageous in describing the ccRCC. In certain embodiments, the methylation of the biomarkers provided in this invention, can be assessed by using the methods described herein. The biomarkers describes can be used in the diagnostic test in order to detect or characterized ccRCC, more specifically to diagnose or to predict ccRCC in an individuals or to evaluate the severity of the disease at the time of diagnosis. The methylation status of ZNF677, TFAP2B, TAC1, FLRT2, ADAMTS19, BMP7 and SIM1 additionally to FBN2, PCDH8 and SFRP1 individualy or in various combinations can be used as diagnostic biomarkers of ccRCC. In particular embodiments, the biomarkers include at least one of ZNF677 (SEQ ID NO: 1), TFAP2B (SEQ ID NO: 4), TAC1 (SEQ ID NO: 5), FLRT2 (SEQ ID NO: 6), ADAMTS19 (SEQ ID NO: 8), BMP7 (SEQ ID NO: 9), SIM1 (SEQ ID NO: 10) as well as FBN2 (SEQ ID NO: 2), PCDH8 (SEQ ID NO: 3) and SFRP1 (SEQ ID NO: 7).

A method for detecting ccRCC in a individual can encompass the steps of: a) obtainment of biological sample from the individuals b) determination of the methylation status of at least one biomarker described herein in the test sample; c) identification of the methylation status of at least one biomarker from the panel of ZNF677 (SEQ ID NOs: 1 and/or 11), TFAP2B (SEQ ID NOs: 4 and/or 14), TAC1 (SEQ ID NOs: 5 and/or 15), FLRT2 (SEQ ID NOs: 6 and/or 16), ADAMTS19 (SEQ ID NO: 8), BMP7 (SEQ ID NO: 9), SIM1 (SEQ ID NO: 10) as well as FBN2 (SEQ ID NOs: 2 and/or 12), PCDH8 (SEQ ID NOs: 3 and/or 13) and SFRP1 (SEQ ID NO: 7), wherein the methylation status of discribed biomarkers indicate presence of ccRCC or increased risk of ccRCC development. In one embodiment, the biomarkers methylation status can be analysed by MSP in a DNA samples from renal tissues, urine, or cells. In another embodiment, the DNA methylation status of the biomarker can be analysed by means of QMSP in DNA samples from renal tissue, urine, or cells.

The value of a diagnostic test to properly identify ccRCC can be evaluated by calculation of the assay sensitivity, specificity, accuracy and by performing Receiver operating characteristic (ROC) analysis in order to estimate the area under the curve. The sensitivity refer to the percentage of true positives that are predicted by a test as positives, whereas the specificity refer to the percentage of true negatives that are predicted as negatives. The accuracy refer to the percentage of true positives and true negatives relative to all samples tested. A ROC curve describes sensitivity as a function of [100%-specificity], where the larger value of AUC reflect the more powerful diagnostic value of a test. The methylation status of more than one biomarker for the diagnosis of ccRCC, can be interpreted in various ways. In the one hand the status of the set of biomarkers can be assumed as methylated if at leas one of the biomarker, included in the panel, defined as methylated. On the other hand the estimates of the methylation levels of the biomarkers may be combined by any appropriate mathematical methods (e.g. logistic regression), well-known for the skilled artisan.

Biomarkers for ccRCC Prognosis

In another embodiments, the invention provide tools and methods for predicting the risk of ccRCC progression in an individuals diagnosed with ccRCC. The methylated status and/or the higher methylation levels of the biomarkers described herein refer to unfavorable pathology and progression of ccRCC, while unmethylated status and/or lower methylation level of the biomarkers are associated to the less agressive disease.

The the risk of ccRCC progression can be evaluated by assessing the methylation status of at least one of the biomarker, including ZNF677 (SEQ ID NOs: 1 and/or 11), TFAP2B (SEQ ID NOs: 4 and/or 14), TAC1 (SEQ ID NOs: 5 and/or 15), FLRT2 (SEQ ID NOs: 6 and/or 16), ADAMTS19 (SEQ ID NO: 8), BMP7 (SEQ ID NO: 9), SIM1 (SEQ ID NO: 10). Additionally, one or more known biomarkers from the group, consisting of FBN2 (SEQ ID NOs: 2 and/or 12), PCDH8 (SEQ ID NOs: 3 and/or 13) and SFRP1 (SEQ ID NO: 7) may be included. In the prefered embodiment the biomarkers methylation status is assesed in a DNA sample from renal tissue, urine, or cells. The methylated status of at least one biomarker in the particular biomarker panel can be interpreted as methylated status of that biomarker panel. For instance, the biomarker panels can consist of a) ZNF677 and FBN2; b) ZNF677 and SFRP1; c) ZNF677 and BMP7; d) ZNF677, FBN2 and BMP7; e) ZNF677, PCDH8 and FLRT2; f) ZNF677, PCDH8, FLRT2 and BMP7; g) ZNF677, PCDH8, FLRT2 and SIM1; etc.

In other prefered embodiment, the severity of ccRCC can be evaluated by estimating the methylation level of one or more biomarkers, including ZNF677, FBN2, PCDH8, TFAP2B, TAC1 and FLRT2 identified by SEQ ID NOs: 11-16. In the preferred embodiment, the methylation levels of the biomarkers are assessed in the DNA samples, extracted from the urine samples obtained from the patients, diagnosed with ccRCC.

A method for evaluating the severity of ccRCC or the risk of disease progression encompass the steps of: a) obtainment of biological sample from the individuals; b) determination of the methylation status and/or methylation level of at least one biomarker described herein in the test sample; c) identification of the methylation status and/or methylation level of at least one biomarker from the panel of ZNF677 (SEQ ID NOs: 1 and/or 11), TFAP2B (SEQ ID NOs: 4 and/or 14), TAC1 (SEQ ID NOs: 5 and/or 15), FLRT2 (SEQ ID NOs: 6 and/or 16), ADAMTS19 (SEQ ID NO: 8), BMP7 (SEQ ID NO: 9), SIM1 (SEQ ID NO: 10) as well as FBN2 (SEQ ID NOs: 2 and/or 12), PCDH8 (SEQ ID NOs: 3 and/or 13) and SFRP1 (SEQ ID NO: 7). In one embodiment, the biomarkers methylation status can be analysed by MSP in a DNA samples from renal tissues, urine, or cells. In another embodiment, the DNA methylation status of the biomarker can be analysed by means of QMSP in DNA samples from renal tissues, urine, or cells.

Biomarkers for the Detection of Small Renal Masses and Active Surveillance of their

In certain embodiments, the described invention provides tools and methods for the detection and active surveillance of the patients, diagnosed with small renal masses (SRM). In certain embodiments, the SRM assessment by means of the present invention, can indicate the need of discontinue the active surveillance and/or to start an active treatment of the individual diagnosed with SRM. Alternatively, a result reflecting low risk of progression can suggest, that there are no needs of active treatment and/or that active surveillance of the individual can be continued.

The detection of SRM in the individuals and monitoring of their can be pursued by evaluating the methylation levels or status of one or more biomarkers from the group consisting of ZNF677, FBN2, PCDH8, TFAP2B, TAC1 and FLRT2 according to SEQ ID NOs: 11-16. In the preferred embodiment, the methylation levels and/or methylation status of the biomarkers are assessed in the DNA samples, extracted from the urine samples obtained from the individuals diagnosed with SRM, since samples of renal tissue are generally unobtainable due to fear of spread of tumour cells along the biopsy tract. In certain embodiments, the differences of the methylation levels and/or status can be compared between the serial samples of patients with SRM. The higher methylation level and/or increased methylation incidence are associated with the presence of SRM and/or progression of the disease and, thus, can be used to identify the ongoing pathological process and to consider the discontinuation of the active surveillance and/or initiation of the active treatment.

A method for detection and/or monitoring of SRM in the individuals encompass the steps: a) obtainment of biological sample (urine) from the individuals; b) determination of the methylation level and/or status of at least one biomarker described herein in the test sample; c) identification of the methylation level and/or status of at least one biomarker from the panel including ZNF677, FBN2, PCDH8, TFAP2B, TAC1 and FLRT2 according to SEQ ID NOs: 11-16. In one embodiment, the DNA methylation status of the biomarker can be analysed by means of QMSP in DNA samples from urine obtained from individuals with SRM.

### EXAMPLES

### Materials and Methods

ccRCC Cases

Fresh-frozen renal tissue samples from patients diagnosed with clear cell renal cell carcinoma were collected between 2013 and 2016 from 123 patients who underwent partial or radical nephrectomy at the Urology Centre of Vilnius University Hospital “Santaros Klinikos”. Noncancerous renal tissue (NRT) samples were available from 45 ccRCC patients as a control group. All tissues were sampled and evaluated by an expert pathologist.

Voided urine samples were collected from the same 123 patients diagnosed with clear cell renal cell carcinoma, and from 93 asymptomatic cases (ASC). All urine samples were centrifuged at 2000 rcf for 15 min at room temperature (Hettich® Universal 320R Centrifuge, DJB Labcare, Buckinghamshire, United Kingdom), supernatant was removed, and sediments were washed twice with 1× PBS. Samples were stored at −80° C. until use.

None of these patients had preoperatively received cancer therapy. Patient and sample data are provided in Table 1.

TABLE 1
Demographic and clinical-pathological characteristics of the patients
with clear cell renal cell carcinoma according to the analysis groups.

| Parameter | | | | | | | |
|---|---|---|---|---|---|---|---|
| Methylation | | | Methylation | | | Gene expression | |
| analysis group | | | analysis group | | | analysis group | |
| (tissues) | | | (urine) | | | (tissues) | |
| Group composition | | | | | | | |
| ccRCC | ccRCC* | NRT | ccRCC | ccRCC* | ASC | ccRCC | NRT |
| (N = 123) | (N = 107) | (N = 45) | (N = 123) | (N = 107) | (N = 93) | (N = 120) | (N = 45) |

Age, years

| | | | | | | | | |
|---|---|---|---|---|---|---|---|---|
| Mean ± SD, | 63 ± 12 | 63 ± 12 | 63 ± 15 | 63 ± 12 | 63 ± 12 | 60 ± 9 | 63 ± 13 | 63 ± 15 |
| [min; max] | [21; 85] | [21; 85] | [21; 85] | [21; 85] | [21; 85] | [27; 82] | [21; 85] | [21; 85] |

Gender

| | | | | | | | | |
|---|---|---|---|---|---|---|---|---|
| Male | 64 (52%) | 55 (51%) | 20 (44%) | 64 (52%) | 55 (51%) | 49 (53%) | 62 (52%) | 20 (44%) |
| Female | 59 (48%) | 52 (49%) | 25 (56%) | 59 (48%) | 52 (49%) | 44 (47%) | 58 (48%) | 25 (56%) |

Stage

| | | | | | | | | |
|---|---|---|---|---|---|---|---|---|
| pT1 | 52 (42%) | 48 (45%) | — | 52 (42%) | 48 (45%) | — | 51 (43%) | — |
| pT2 | 5 (4%) | 5 (5%) | — | 5 (4%) | 5 (5%) | — | 5 (4%) | — |
| pT3 | 61 (50%) | 51 (47%) | — | 61 (50%) | 51 (47%) | — | 59 (49%) | — |
| pT4 | 5 (4%) | 3 (3%) | — | 5 (4%) | 3 (3%) | — | 5 (4%) | — |

Tumor size, mm

| | | | | | | | | |
|---|---|---|---|---|---|---|---|---|
| Mean ± SD, | 57 ± 29 | 54 ± 28 | — | 57 ± 29 | 54 ± 28 | — | 57 ± 29 | — |
| [min; max] | [20; 180] | [20; 180] | | [20; 180] | [20; 180] | | [20; 180] | |

Fuhrman grade

| | | | | | | | | |
|---|---|---|---|---|---|---|---|---|
| 1 | 4 (3%) | 3 (3%) | — | 4 (3%) | 3 (3%) | — | 4 (3%) | — |
| 2 | 46 (37%) | 43 (40%) | — | 46 (37%) | 43 (40%) | — | 45 (38%) | — |
| 3 | 64 (52%) | 55 (51%) | — | 64 (52%) | 55 (51%) | — | 63 (52%) | — |
| 4 | 6 (5%) | 3 (3%) | — | 6 (5%) | 3 (3%) | — | 5 (4%) | — |
| Unknown | 3 (3%) | 3 (3%) | — | 3 (3%) | 3 (3%) | — | 3 (3%) | — |

Differentiation grade

| | | | | | | | | |
|---|---|---|---|---|---|---|---|---|
| 1 | 6 (5%) | 4 (4%) | — | 6 (5%) | 4 (4%) | — | 6 (5%) | — |
| 2 | 71 (58%) | 66 (62%) | — | 71 (58%) | 66 (62%) | — | 69 (58%) | — |
| 3 | 46 (37%) | 37 (34%) | — | 46 (37%) | 37 (34%) | — | 45 (37%) | — |

Intravascular invasion

| | | | | | | | | |
|---|---|---|---|---|---|---|---|---|
| No | 83 (67%) | 78 (73%) | — | 83 (67%) | 78 (73%) | — | 82 (68%) | — |
| Yes | 40 (33%) | 29 (27%) | — | 40 (33%) | 29 (27%) | — | 38 (32%) | — |

Fat invasion

| | | | | | | | | |
|---|---|---|---|---|---|---|---|---|
| No | 73 (59%) | 65 (61%) | — | 73 (59%) | 65 (61%) | — | 72 (60%) | — |
| Yes | 50 (41%) | 42 (39%) | — | 50 (41%) | 42 (39%) | — | 48 (40%) | — |

Tumor necrosis

| | | | | | | | | |
|---|---|---|---|---|---|---|---|---|
| No | 94 (76%) | 83 (76%) | — | 94 (76%) | 83 (76%) | — | 92 (77%) | — |
| Yes | 29 (24%) | 24 (24%) | — | 29 (24%) | 24 (24%) | — | 28 (23%) | — |

*For the Kaplan-Meier survival and cox proportional hazard regretion analysis.
ccRCC—clear cell renal cell carcinoma, NRT—noncancerous renal tissue, ASC—asymptomatic control, SD—standard deviation.

### SRM Cases

In this prospective part of the study, patients were included at the time of SRM detection (0 mo) and after 6, 12, 18, 24 months of active surveillance (6 mo, 12 mo, 18 mo, 24 mo), N=20 patients in total. The individuals were included between March 2018 and July 2020, and examined at the National Cancer Institute (Vilnius, Lithuania). Clinical data were extracted from patients' records by staff clinicians and provided in Table 2. Urine samples were collected at different time points with regard to the examination.

TABLE 2
Demographic and clinical-pathological characteristics
of the patients with small renal masses.

| Parameter | Active surveillance time points | | | | |
|---|---|---|---|---|---|
| Group | 0 mo | 6 mo | 12 mo | 18 mo | 24 mo |
| composition | (N = 20) | (N = 17) | (N = 15) | (N = 9) | (N = 5) |

Age at diagnosis, years

| | | | | | |
|---|---|---|---|---|---|
| Mean ± SD, | 75 ± 10 | 78 ± 9 | 78 ± 9 | 76 ± 6 | 77 ± 3 |
| [min; max] | [59; 93] | [62; 93] | [62; 93] | [62; 80] | [73; 80] |

Gender

| | | | | | |
|---|---|---|---|---|---|
| Male | 12 (60%) | 10 (59%) | 8 (53%) | 6 (67%) | 4 (80%) |
| Female | 8 (40%) | 7 (41%) | 7 (47%) | 3 (33%) | 1 (20%) |

Tumour size, mm

| | | | | | |
|---|---|---|---|---|---|
| Mean ± SD, | 24 ± 8 | 25 ± 8 | 26 ± 9 | 28 ± 12 | 38 ± 7 |
| [min; max] | [14; 37] | [11; 39] | [14; 40] | [15; 49] | [31; 45] |

Stage at diagnosis

| | | | | | |
|---|---|---|---|---|---|
| pT1a | 19 (95%) | 16 (94%) | 12 (80%) | 6 (67%) | 2 (40%) |
| pT1b | — | — | 1 (7%) | — | 1 (20%) |
| Unknown | 1 (5%) | 1 (6%) | 2 (13%) | 3 (33%) | 2 (40%) |

Differentiation grade at diagnosis

| | | | | | |
|---|---|---|---|---|---|
| 1 | 4 (20%) | 4 (23%) | 2 (13%) | 2 (22%) | 2 (40%) |
| 2 | 14 (70%) | 12 (71%) | 12 (80%) | 6 (67%) | 3 (60%) |
| Unknown | 2 (10%) | 1 (6%) | 1 (7%) | 1 (11%) | — |

mo—months; SD—standard deviation.

### Genome-Wide DNA Methylation Profiling

For the initial screening, the genome-wide DNA methylation profiling of 11 paired ccRCC and NRT samples was performed in order to identify potential ccRCC biomarkers. The samples were processed using the two-colour Human DNA Methylation 1×244K Microarrays (Agilent Technologies, Santa Clara, CA, USA), according to the manufacturer's protocol. Saturated, non-uniform and outlier probe signals were treated as compromised and removed from the analysis. Normalized log ration (Cy5/Cy3) representing methylated/reference DNA was used for further calculations. Probe annotations were uploaded from the SureDesign platform (https://earray.chem.agilent.com/suredesign). Group comparison-specific filtering leaving only probes detected in 100% of samples was performed. For group comparison, fold change (FC) values were estimated and paired or unpaired t-test was applied. Analysis were performed with GeneSpring GX v14.9 software (Agilent Technologies).

The gene set enrichment analysis (GSEA) for the identified differentially methylated genes between groups was performed using publicly available online GSEA tool and Molecular Signatures Database (MSigDB, v5.2; http://software.broadinstitute.org/gsea), both maintained by Broad Institute (Cambridge, MA, USA).

### DNA Purification

Renal issue samples were put in liquid nitrogen and mechanically homogenized into powder using cryoPREP™ CP02 Impactor with tissue TUBE TT1 (Covaris, Woburg, MA, USA). Total volume of urine were centrifuged at 2000 rcf for 15 min and supernatant was removed, while urine sediments resuspended with 2 mL of 1xPBS and centrifuged at the same conditions two more times. Homogenized tissue powder and/or urine sediments were treated for up to 18 h at 55° C. with 10-25 μl of proteinase K (Thermo Scientific™, Thermo Fisher Scientific, Wilmington, DE, USA) and 500 μl of lysis buffer, consist of 50 mM Tris-HCl PH 8.5, 1 mM EDTA, 0.5% Tween-20 (all from Carl Roth, Karlsruhe, Germany) for tissue samples and 10 mM Tris-HCl pH 8.0, 1% SDS, 75 mM NaCl (all from Carl Roth) for urine samples. DNA was extracted following the standard phenol-chloroform purification and ethanol precipitation protocol. The concentration and purity of the extracted DNA were measured by NanoDrop™ 2000 spectrophotometer (Thermo Scientific™).

### Bisulfite Conversion

For DNA methylation analysis using qualitative or quantitative methylation-specific PCR (MSP or QMSP, respectively), 400 ng of purified DNA were modified with bisulfite, using EZ DNA Methylation™ Kit (Zymo Research, Irvine, CA, USA) according to the manufacturer's protocol. However, different from the protocol, the initial step was performed for 15 min at 42° C. For the elution, 40 μl of elution buffer was used. Prepared DNA samples were analysed immediately or stored at −20° C. The DNA sequences of fully methylated biomarkers after modifikation using bisulfite are provided in Table 3.

TABLE 3
The amplicon sequences of the DNA methylation biomarkers.

| | | |
|---|---|---|
| Assay | Sequence ID | Fully methylated sequence (5′→3′) |

MSP amplicons

| | | |
|---|---|---|
| ZNF677 | SEQ ID NO. 1 | TCGCGGGTTATAGGTTTTTACGTTTCGTTGTCGGGTTTTGGGGTCGTTTTGTAGGTTAA |
| | | AATTTCGAATTTGTTTATTTTTTTCGCGGCGTGGTTTTAAGACGTTTTTAGTTTCGTCGT |
| | | TTCGAGAGGGTTTAGAGATTTGCGTTTATTTCGGATT |
| FBN2 | SEQ ID NO. 2 | TTTAATATTOGTTTTCGGAGCGTACGGGAATTCGTCGAGTTTTGCGTGTAGGTTTTTTTT |
| | | TTTTTTGAGGTTTATATTTTTTGAAATTTTACGTTAGGGTTTTTGTAATTTTTTTTTTCGT |
| | | TCGTTGACGGTTTTGGAGTCGTTCGGGGTTTTAGGTCGGTTATGTAACGTGTATCGTTC |
| | | GG |
| PCDH8 | SEQ ID NO. 3 | TTTAGAGTTCGTTGGAGGTTCGGAGTTGTTATTCGTAGATTTTTTTCGTATAGGGTTCGT |
| | | AAAGAGCGTGATTTCGAGAGTTTGAGATTGACGTTCGATTTGGAAATTAGAGAAGATTTTT |
| | | TTAGTTTTTCGGATCGTATTTGAGG |
| TFAP2B | SEQ ID NO. 4 | TTCGAAGATTTTAAGAGTGGGCGATTTATAGGCGCGGTCGGTAAGTTTTTGGGGGATTC |
| | | GGGTTCGGACGAGCGTTTATAGGTAGCGTTT |
| TAC1 | SEQ ID NO. 5 | GGTATTGAGTAGGCGAAAGAGCGCGTTCGGATTTTTTTTTCGGCGGTAGTTATCGAGAG |
| | | TGCGGAGCGATTAGCGTGCGTTCGGAGGAATTAGAGAAATTTAGTATTTCGGGGGATTG |
| | | TTCGTCGTAGTAAGTGTTCGC |
| FLRT2 | SEQ ID NO. 6 | TAGTATTTGGAGCGAGTTTTGCGTTCGTTTTCGCGTAGCGTCGTACGTTCGGTTTCGAG |
| | | TTGTTCGTATATACGCGTCGGAGGAGAGTTCGTTTAGTTTTTTCGTCGAGTTTCGGGAT |
| | | TTTTTAAATTCGAGGAGTTTCGGCGTCGCGGGGTAGTTTTTTGTCGTTTTTTTCGTTCG |
| | | TTGTATTTTTTTTGGGGTTCGTTGGTTTGGCGAAGCGGAGAGGGGGAGGCGGAGGAGG |
| | | AGAGAAGGGGGGGGTCGCGGCGGTCGAAGTTAAGAGAAAGTG |
| SFRP1 | SEQ ID NO. 7 | TCGCGTTTGGTTTTAGTAAATCGAATTCGTTCGCGAGGGAGGCGATTGGTTTTCGCGTC |
| | | GGTGACGGACGTGGTAACGAGTGCGGTTCGTTTCGTCGGGAGTTGATTGGTTGCGCGG |
| | | GGCGGTTTCGAGGGTTCGGTCGTAGGAGTTTCGCGTATT |
| ADAMTS19 | SEQ ID NO. 8 | AAAGGGTTTGGGTAAATTCGTCGTTCGGTTTTTTAGCGTTTCGGGGAGGTCGTTGCGTT |
| | | TCGGAGTGGATCGCGTTGGAGGCGTGCGTCGGGCGAGAAGTCGCGGTCGCGGGAGCG |
| | | TAGTATGGGGAAGAATCGCGAGATGCGTTTGATTTATATTT |
| BMP7 | SEQ ID NO. 9 | GTTTTTTAAGTTTTGCGGTGCGTTCGGGGGAGTGCGGGGCGAGGGGTTCGGGGTTAGT |
| | | ATCGAGTAGGGGGGGGGGGTTCGGGTAGAGCGCGGTCGGTCGGGGAGGGGTTATGTT |
| | | TGGCGCGGGCGTAGCGGGGTTCGTTTGTAGTAAGTGATCGAGCGGC |
| SIM1 | SEQ ID NO. 10 | GTGAAGTAGAAGACGTTTCGCGTTCGTTTAGTAGTTTCGTAGTTTCGCGGTGGTGTGGG |
| | | AGAGGTCGCGGCGTTTTTTATTTTCGGGGGAGTTTGCGAGGGGTTGTCGCGAGCGCGT |
| | | TATTTGTTAATTT |

QMSP amplicons

| | | |
|---|---|---|
| ZNF677 | SEQ ID NO. 11 | GGCGTTTTCGGGTGAGTTTTCGTTTTTCGGGTTTAAGTTTGCGTTTCGGGGGTTATAGG |
| | | TTTTTACGTTTCGTTGTCGGGTTTTGGGGTCGTTTTG |
| FBN2 | SEQ ID NO. 12 | TGACGGTTTTGGAGTCGTTCGGGGTTTTAGGTCGGTTATGTAACGTGTATCGTTCGGGG |
| | | TTGTCGGTTGTATTTTCGTCGCGTTTCGTCGTTTATTGCGTTA |
| PCDH8 | SEQ ID NO. 13 | TAGAGTGAGGGGGGGTTCGCGCGTTTTTAGAGTTCGTTGGAGGTTCGGAGTTGTTATTC |
| | | GTAGATTTTTTTCGTATAGGGTTCGTAAAGAG |
| TFAP2B | SEQ ID NO. 14 | CGGGATAGTTTTTGAAAGTTCGGCGTAGAGTCGTTTCGAAGATTTTAAGAGTGGGCGATT |
| | | TATAGGCGCGGTCGGTAAGTTTTTGGGGGATTCGGGTTCGGACGAGCGTTTATAGGTA |
| TAC1 | SEQ ID NO. 15 | GAGCGATTAGCGTGCGTTCGGAGGAATTAGAGAAATTTAGTATTTCGCGGGATTGTTCGT |
| | | CGTAGTAAGTGTTCGCGCGGTGTTGGTCGCGGTTGTTCGGGTTATTT |
| FLRT2 | SEQ ID NO. 16 | AGTTTTTAGATTTACGTCGGGGGGGGGTAGTATTTGGAGCGAGTTTTGCGTTCGTTTTC |
| | | GCGTAGCGTCGTACGTTCGGTTTCGAGTTGTTC |
| ACTB* | SEQ ID NO. 17 | AACCAATAAAACCTACTCCTCCCTTAAAAATTACAAAAACCACAACCTAATAAAAAAAATAACC |
| | | ACCACCCAACACACAATAACAAACACAAATTCACAATCCAAAAAACTTACTAAACCTCCTCCA |
| | | TCACCA |

*-endogenous control.
Abbreviations:
MSP-methylation-specific PCR, QMSP-quantitative methylation-specific PCR.

### Qualitative Methylation-Specific PCR

The bisulfite converted DNA were used as template for MSP. The MSP primers for unmetylated and methylated DNA for genes ZNF677, FBN2, PCDH8, TRAP2B, TAC1, FLRT2, SFRP1, ADAMTS19, BMP7 and SIM1 were designd using Methyl Primer Express® Software v1.0 (Applied Biosystems™, Thermo Fisher Scientific, Carlsbad, CA, USA) and ordered from Metabion (Martinsried, Germany) (Table 4). The reaction mix of MSP (25 μl in totall) consisted of 1x PCR Gold Buffer, 2.5 mM MgCl2, 0.4 mM of each dNTP, 1.25 U AmpliTaq Gold® 360 DNA Polymerase (Applied Biosystems™, Thermo Scientific™), 1 μL of 360 GC Enhancer, 1 UM of each primer, and ˜10 ng of the bisulfite-treated DNA. Prior the analysis, the reaction conditions were optimised and consisted of 10 min at 95° C., 34-38 cycles of 45 s at 95° C., primer annealing for 45 s at 58-65° C. (Table 4) and elongation for 45 s at 72° C., followed by 5-10 min at 72° C. For each primer pair, methylated control (MC), unmethylated control (UC) and no-template control (NTC) were included in all MSP sassays. Obtained amplification products (provided in Table 3 for methylated DNA) were analysed in 3% agarose gel. For UC, bisulfite converted human leucocyte DNA awas used, meanwhile CpG methyltransferase-treated (Thermo Scientific™) and bisulfite converted human leukocyte DNA served as the MC. Only runs, wherein the UC provided a product with primers specific only for unmethylated DNA, the MC provided a product with primers specific only for methylated DNA and the NTC gave no apmlification product, was considered as valid. The individual biomarker was considered as methylated if the amplification product in the sample with primers, specific for methylated DNA was detected. A biomarker was considered as unmethylated if the amplification product in the sample with primers, specific only for unmethylated DNA was detected and there is no amplification product with the primers specific for the methylated DNA.

TABLE 4
Qualitative methylation-specific PCR (MSP) primers, used for the assays, and
amplification conditions.

| | | | | | Primer | Number |
|---|---|---|---|---|---|---|
| | | Primer/probe sequence | Primer | Amplicon | annealing | of MSP |
| Assay | Sequence ID | (5′→3′) | type | size, nt | t° C. | cycles |
| ZNF677 | SEQ ID NO. 18 | TCGGGGGTTATAGGTTTTTAC | M-F | 156 | 58 | 37 |
| | SEQ ID NO. 19 | AATCCGAAATAAACGCAAATCTC | M-R | | | |
| | SEQ ID NO. 20 | GTTTTGTGGGTTATAGGTTTTTATG | U-F | 162 | 58 | |
| | SEQ ID NO. 21 | TTTAATCCAAAATAAACACAAATCTCT | U-R | | | |
| FBN2 | SEQ ID NO. 22 | TTTAATATTCGTTTTCGGAGCG | M-F | 182 | 58 | 37 |
| | SEQ ID NO. 23 | CCGAACGATACACGTTACATAA | M-R | | | |
| | SEQ ID NO. 24 | GTAGTTTTTTAATATTTGTTTTTGGAGTG | U-F | 192 | 58 | |
| | SEQ ID NO. 25 | ACCCCAAACAATACACATTACATAA | U-R | | | |
| PCDH8 | SEQ ID NO. 26 | TTTAGAGTTCGTTGGAGGTTC | M-F | 146 | 58 | 37 |
| | SEQ ID NO. 27 | CCTCAAATACGATCCGAAAAAC | M-R | | | |
| | SEQ ID NO. 28 | GTTTTTAGAGTTTGTTGGAGGTTT | U-F | 152 | 58 | |
| | SEQ ID NO. 29 | CAACCTCAAATACAATCCAAAAAAC | U-R | | | |
| TFAP2B | SEQ ID NO. 30 | TTCGAAGATTTTAAGAGTGGGC | M-F | 90 | 58 | 35 |
| | SEQ ID NO. 31 | AAACGCTACCTATAAACGCTCG | M-R | | | |
| | SEQ ID NO. 32 | GTTTTGAAGATTTTAAGAGTGGGT | U-F | 94 | 58 | |
| | SEQ ID NO. 33 | CCAAACACTACCTATAAACACTCA | U-R | | | |
| TAC1 | SEQ ID NO. 34 | GGTATTGAGTAGGCGAAAGAGC | M-F | 139 | 65 | 35 |
| | SEQ ID NO. 35 | GCGAACACTTACTACGACGAAC | M-R | | | |
| | SEQ ID NO. 36 | TAAGGTATTGAGTAGGTGAAAGAGT | U-F | 143 | 63 | |
| | SEQ ID NO. 37 | CACAAACACTTACTACAACAAACAAT | U-R | | | |
| FLRT2 | SEQ ID NO. 38 | TAGTATTTGGAGCGAGTTTTGC | M-F | 277 | 63 | 38 |
| | SEQ ID NO. 39 | CACTTTCTCTTAACTTCGACCG | M-R | | | |
| | SEQ ID NO. 40 | GTAGTATTTGGAGTGAGTTTTGTGT | U-F | 279 | 63 | |
| | SEQ ID NO. 41 | CCACTTTCTCTTAACTTCAACCA | U-R | | | |
| SFRP1 | SEQ ID NO. 42 | TCGCGTTTGGTTTTAGTAAATC | M-F | 156 | 58 | 36 |
| | SEQ ID NO. 43 | AATACGCGAAACTCCTACGAC | M-R | | | |
| | SEQ ID NO. 44 | GAGTTGTGTTTGGTTTTAGTAAATT | U-F | 161 | 58 | |
| | SEQ ID NO. 45 | AAAATACACAAAACTCCTACAACC | U-R | | | |
| ADAMTS19 | SEQ ID NO. 46 | AAAGGGTTTGGGTAAATTCGTC | M-F | 157 | 58 | 36 |
| | SEQ ID NO. 47 | AAATATAAATCAAACGCATCTCGC | M-R | | | |
| | SEQ ID NO. 48 | TAAAGGGTTTGGGTAAATTTGTTG | U-F | 160 | 58 | |
| | SEQ ID NO. 49 | ACAAATATAAATCAAACACATCTCAC | U-R | | | |
| BMP7 | SEQ ID NO. 50 | GTTTTTTAAGTTTTGCGGTGCG | M-F | 161 | 61 | 35 |
| | SEQ ID NO. 51 | GCCGCTCGATCACTTACTAC | M-R | | | |
| | SEQ ID NO. 52 | GTTGTTTTTTAAGTTTTGTGGTGT | U-F | 165 | 61 | |
| | SEQ ID NO. 53 | CACCACTCAATCACTTACTACA | U-R | | | |
| SIM1 | SEQ ID NO. 54 | GTGAAGTAGAAGACGTTTCGC | M-F | 130 | 62 | 34 |
| | SEQ ID NO. 55 | AAATTAACAAATAACGCGCTCG | M-R | | | |
| | SEQ ID NO. 56 | TAGGTGAAGTAGAAGATGTTTTGT | U-F | 135 | 62 | |
| | SEQ ID NO. 57 | CCAAATTAACAAATAACACACTCAC | U-R | | | |

M/U-primers specific for methylated/unmethylated DNA template after bisulfite modification, F/R-forward/reverse primers.

### Quantitative Methylation-Specific PCR

The bisulfite converted DNA were used as template for QMSP. The QMSP primers and probes specific for methylated DNA for genes ZNF677, FBN2, PCDH8, TRAP2B, TAC1 and FLRT2 were designd using Methyl Primer Express® Software v1.0 (Applied Biosystems™, Thermo Fisher Scientific, Carlsbad, CA, USA) and ordered from Metabion (Martinsried, Germany) (Table 5). All primers and probes overlape at least a fragment of MSP primers. The primers for ACTB, which are not overlapping with CpG dinucleotides, were selected from the previous study and were used in each run for normalization of the DNA input. The sequence of amplification products are provided in Table 3. QMSP was performed in triplicates for each set of primers in separete wells. The reaction mix (20 μl in totall) consisted of 1× TaqMan® Universal Master Mix II, no UNG (Applied Biosystems™), 300 nM of each primer, 50 nM of probe, and ˜10 ng of 20 bisulfite-converted DNA. All assays were carried out under the following conditions: 95° C. for 10 min followed by 50 cycles of 95° C. for 15 s and 60° C. for 1 min, using the ViiA7 qPCR System (Applied Biosystems™). Only runs, wherein MCs provided a positive signal and the NTC gave no apmlification product, was considered as valid.

A run was considered valid when routinely included MCs gave a positive signal and there was no amplification in NTC wells. The background-based threshold algorithm was applied for the estimation of Cq value. The methylation level of the particular biomarker was calculated using the Formula 1 and expressed as a percentage of the methylated reference DNA.

$$Methylation level , \text{} \% = \frac{100 \%}{{2}^{( Cq of X in sample - Cq of ACTB in sample ) - ( Cq of X in MC - Cq of ACTB in MC )}}$$

Formula 1. The formula used for calculating the methylation level of the particular gene (X). The methylation level is expressed in percentage. Cq—cycle of quantification value, MC—methylated (positive) DNA standard (control sample).

TABLE 5
Quantitative methylation-specific PCR (QMSP) primers and probes, used for the
assays.

| | | | Primer/probe | Amplicon |
|---|---|---|---|---|
| Assay | Sequence ID | Primer/probe sequence (5′→3′) | type | size, nt |
| ZNF677 | SEQ ID NO. 58 | GGCGTTTTCGGGTGAGTTTTC | QM-F | |
| | SEQ ID NO. 59 | CAAAACGACCCCAAAACCCG | QM-R | 96 |
| | SEQ ID NO. 60 | FAM-GAAACGTAAAAACCTATAACCCGCGAAACG-BHQ-1 | QM-P | |
| FBN2 | SEQ ID NO. 61 | TGACGGTTTTGGAGTCGTTC | QM-F | |
| | SEQ ID NO. 62 | TAACGCAATAAACGACGAAACG | QM-R | 102 |
| | SEQ ID NO. 63 | FAM-CGACAACCCCGAACGATACACGTTACA-BHQ-1 | QM-P | |
| PCDH8 | SEQ ID NO. 64 | TAGAGTGAGGGGGGTTC | QM-F | |
| | SEQ ID NO. 65 | CTCTTTACGAACCCTATACGAA | QM-R | 91 |
| | SEQ ID NO. 66 | FAM-CGAACCTCCAACGAACTCTAAAAACGCG-BHQ-1 | QM-P | |
| TFAP2B | SEQ ID NO. 67 | CGGGATAGTTTTTGAAAGTTCG | QM-F | |
| | SEQ ID NO. 68 | TACCTATAAACGCTCGTCCG | QM-R | 118 |
| | SEQ ID NO. 69 | FAM-GAGTCGTTTCGAAGATTTTAAGAGTGGGCG-BHQ-1 | QM-P | |
| TAC1 | SEQ ID NO. 70 | GAGCGATTAGCGTGCGTTC | QM-F | |
| | SEQ ID NO. 71 | AAATAACCCGAACAACCGCGA | QM-R | 107 |
| | SEQ ID NO. 72 | FAM-TTGTTCGTCGTAGTAAGTGTTCGCGC-BHQ-1 | QM-P | |
| FLRT2 | SEQ ID NO. 73 | AGTTTTTAGATTTACGTCGGGC | QM-F | |
| | SEQ ID NO. 74 | GAACAACTCGAAACCGAACG | QM-R | 92 |
| | SEQ ID NO. 75 | FAM-GCGAGTTTTGCGTTCGTTTTCGCG-BHQ-1 | QM-P | |
| ACTB | SEQ ID NO. 76 | TGGTGATGGAGGAGGTTTAGTAAGT | QM-F | |
| | SEQ ID NO. 77 | AACCAATAAAACCTACTCCTCCCTTAA | QM-R | 133 |
| | SEQ ID NO. 78 | FAM-ACCACCACCCAACACACAATAACAAACACA-BHQ-1 | QM-P | |

Abbreviations:
QM-F/R-forward/reverse primer; QM-P-probe, FAM-fluorescein, BHQ1/3-black hole quencher-1/3.

RNA Extraction and cDNA Synthesis

Total RNA was extracted from the analysed tissue samples by MirVana™ miRNA Isolation Kit (Ambion®, Thermo Fisher Scientific, Foster City, CA, USA) following the manufacturer's protocol and used for targeted gene expression analysis by means of quantitative PCR (qPCR). Briefly, homogenized tissue powder was treated with 500 μL Lysis/Binding Buffer for 10 min on ice, and 50 μL of miRNA Homogenate Additive for additional 10 min. The total RNA was extracted with 500 μL of acid-phenol: chloroform and purified with supplied Filter Cartridges. To eluate purified RNA, 100 μL of 95° C. Elution Solution was used. Samples were used immediately or stored at −40° C. until further use.

### Transcriptional Gene Expression Analysis

For qPCR, 100 ng of the RNA were reverse transcribed (RT) using Maxima First Strand cDNA Synthesis Kit with ds DNase according to the recommended protocol (Thermo Fisher Scientific). Expression of the genes ZNF677, FBN2, PCDH8, TFAP2B, TAC1, FLRT2 and endogenous control HPRT1 was evaluated using TaqMan® Gene Expression Assays (Hs00737026_m1, Hs00266592_m1, Hs00159910_m1, Hs01560931_m1, Hs00243225_m1, Hs00544171_s1 and Hs02800695_m1, respectively; Applied Biosystems™) in duplicates per gene. The reaction mix (20 UL in total) consisted of 1x TaqMan® Universal Master Mix II, no UNG (Applied 20 Biosystems™), 0.6 μL of TaqMan® assay, and 2 μL of RT reaction product. Amplification was performed using ViiA7 qPCR System (Applied Biosystems™) under following thermal cycling conditions: 40 cycles of 95° C. for 15 s and 60° C. for 1 min. NTCs (No-template control) were included in each RT-qPCR run. Relative gene expression values in a linear scale were used for the analysis of the results, performed with GenEx v6.0.1 software (MultiD Analyses AB, Göteburg, Sweden).

### Statistical Analysis

Statistical analyses were performed using STATISTICA™ v8.0 (StatSoft, Tulsa, OK, USA) and MedCalc® v14.0 software (MedCalc Software, Ostend, Belgium). All quantitative variables were tested for normality (Shapiro-Wilk, Kolmogorov-Smirnov and Lilliefors tests) and because of abnormal distribution, nonparametric Wilcoxon-Mann-Whitney test were applied to compare variables between two groups. Meanwhile, 2-sided Fisher's exact test was applied for comparison of categorical variables. Spearman's (RS) 15 rank correlation coefficients were calculated to test the associations between two quantitative variables. Biomarker performance was evaluated by analysing Receiver operating curves (ROC) and calculating the area under the curve (AUC). Biomarkers were also evaluated by calculating various diagnostic test selectivity parameters: sensitivity, specificity, accuracy, positive predictive value (PPV), negative predictive value (NPV), positive likelihood ratio (LR+), negative likelihood ratio (LR-) and Youden index. For time-event analysis, Kaplan-Meier analysis was used to calculate survival estimates. The Cox proportional hazards modelling was performed to estimate the hazard ratio (HR) of death with 95% confidence intervals (CI). After univariate analysis, Cox proportional hazards regression models were used to select variables with P values lower than 0.0500 for the multivariate analysis.

### RESULTS

### Microarray-Based DNA Methylation Profiling for Biomarker Discovery

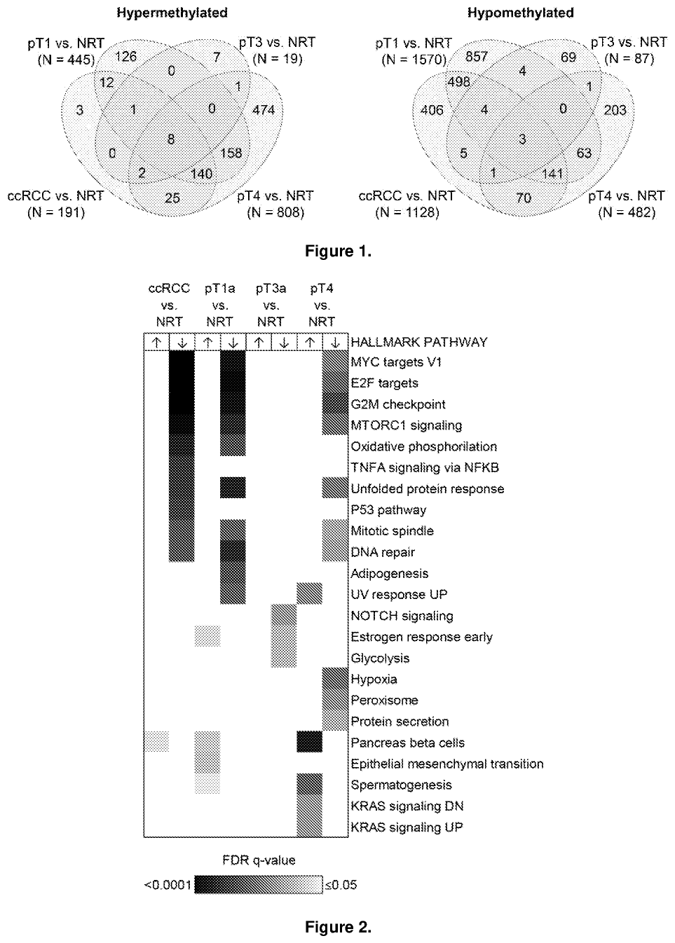

In order to identify potential DNA methylation biomarkers of ccRCC and to determine the amount of DNA methylation changes in cancerous renal tissues, compared to NRT, the genome-wide DNA methylation profile was analysed in 11 pairs of ccRCC and NRT samples. Among ccRCC, 4, 4 and 3 cases were pT1, pT3 and pT4 stages respectively. The comparison of cancerous and non-cancerous renal tissue samples revealed significant methylation differences (fold-change (FC) ≥1.5; P≤0.050) were associated with 1319 genes, of which 191 (14.5%) were hypermethylated and 1128 (85.5%) were hypomethylated (FIG. 1.). According to Benjamini-Hochberg adjusted P value, significant DNA methylation differences were observed in 394 genes, of which 191 (48.5%) were hypermethylated and 203 (51.5%) were hypomethylated. The number of hypermethylated genes in promoter region was a little bit higher than the number of hypomethylated genes (56.9% vs. 43.1%) while both events were similarly common in intragenic loci (50.1% vs. 49.9%). In the comparison of cases with different tumour stages separately with NRT, the most frequent DNA methylation differences were observed in pT1 tumours overlapped with 2007 genes in total of which 437 (21.8%) were hypermethylated and 1562 (77.8%) were hypomethylated including 8 genes (0.4%) with concurrent changes observed according to different microarray probes. The least abberantly methylated genes were found in pT3 stage tumour samples-106 of which 19 (17.9%) were hypermethylated and 87 (82.1%) hypomethylated. In the late pT4 tumour stage samples DNA methylation differences have been identified in 1288 genes, of which 806 (62.6%) were hypermethylated and 480 (37.3%) hypomethylated including 2 (0.2%) overlapped genes. In the case of pT1 and pT3 stages tumour samples, the number of hypomethylated genes in promoter (75.7% and 74.3% respectively) and intragenic (76.9% and 85.3% respectively) regions was much higher than the number of hypermethylated genes (pT1:24.3% and 23.1%; pT3: 25.7% and 14.7% respectively). On the contrary in pT4 stage tumour samples, hypermethylation in the promoter and intragenic regions was higher (63.1% and 68.8% respectively) than hypomethylation (36.9% and 31.2% respectively).

According to the GSEA analysis, gene sets involved in cell cycle regulation and apical junctions were among the most significantly enriched in ccRCC samples compared to NRT cases. The increase of methylation levels was the most significant among the genes involved in epithelial-mesenchymal transition (EMT), early estrogen response and down regulated in KRAS signaling, meanwhile decreased methylation was commonly observed in genes involved in cell cycle regulation, DNA repair, upregulated in response to ultraviolet (UV), hypoxia, WNT-B catenin and NOTCH signaling and others (FIG. 2)

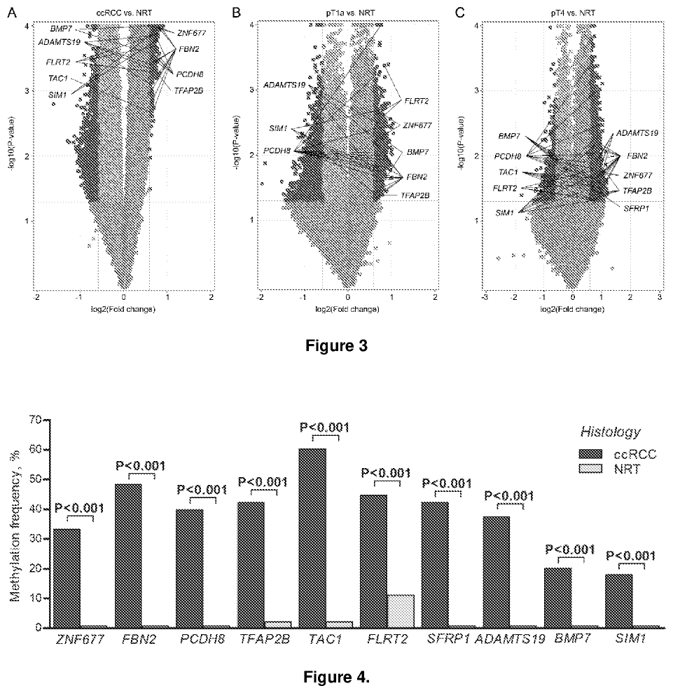

Based on methylation differences according to the renal tissue histology as observed in DNA methylome profiling data and with regard to the GSEA analysis, 10 genes-ZNF677, FBN2, PCDH8, TFAP2B, TAC1, FLRT2, SFRP1, ADAMTS19, BMP7 and SIM1 were selected as biomarkers for further analysis (FIG. 3).

### Qualitative DNA Methylation Analysis at Regulatory Regions of the Selected Putative Biomarkers

DNA methylation status of ZNF677, FBN2, PCDH8, TFAP2B, TAC1, FLRT2, SFRP1, ADAMTS19, BMP7 and SIM1 was analysed qualitatively at regulatory regions of the genes. 123 ccRCC and 45 NRT samples (Table 1) were investigated by means of MSP using the primers
provided in Table 4. Methylation of FBN2, PCDH8 and SFRP1 was previously reported to be frequently methylated in ccRCC tissues [29-31] nevertheless was also icluded in the analysis.

Methylation of ZNF677, FBN2, PCDH8, TFAP2B, TAC1, FLRT2, SFRP1 and ADAMTS19 was detected at least in one third of ccRCC samples or even more frequently (from 33.3% to 60.2%), while less common methylation events was observed in BMP7 and SIM1 (20.3% and less). Methylation of the selected genes was significantly more common in cancerous renal tissues as cmpared to NRT (from 0.0% to 11.1%; all P<0.0100; FIG. 4). The separate biomarkers had high specificity (288.9%) and positive predictive values (≥90.4%) (Table 6).

TABLE 6
The diagnostic test performance characteristics of the analysed methylation biomarkers in renal tissues.

| | | | | | | | | | |
|---|---|---|---|---|---|---|---|---|---|
| | Sensitivity, | Specificity, | Accuracy, | | | | | | Younden |
| VARIABLE | % | % | % | PPV, % | NPV, % | LR+ | LR− | AUC | index |

Single gene

| | | | | | | | | | |
|---|---|---|---|---|---|---|---|---|---|
| ZNF677 | 33.33 | 100.00 | 53.33 | 100.00 | 39.13 | — | 0.67 | 0.67 | 0.333 |
| FBN2 | 48.36 | 100.00 | 63.85 | 100.00 | 45.35 | — | 0.52 | 0.74 | 0.484 |
| PCDH8 | 39.84 | 100.00 | 57.89 | 100.00 | 41.60 | — | 0.60 | 0.70 | 0.398 |
| TFAP2B | 42.28 | 97.78 | 58.93 | 97.80 | 42.06 | 19.02 | 0.59 | 0.70 | 0.401 |
| TAC1 | 60.16 | 97.78 | 71.45 | 98.44 | 51.27 | 27.07 | 0.41 | 0.79 | 0.579 |
| FLRT2 | 44.72 | 88.89 | 57.97 | 90.38 | 40.80 | 4.024 | 0.62 | 0.67 | 0.336 |
| SFRP1 | 42.28 | 100.00 | 59.59 | 100.00 | 42.61 | — | 0.58 | 0.71 | 0.423 |
| ADAMTS19 | 37.40 | 100.00 | 56.18 | 100.00 | 40.64 | — | 0.63 | 0.69 | 0.374 |
| BMP7 | 20.33 | 100.00 | 44.23 | 100.00 | 34.98 | — | 0.80 | 0.60 | 0.203 |
| SIM1 | 17.89 | 100.00 | 42.52 | 100.00 | 34.29 | — | 0.82 | 0.59 | 0.179 |

PPV—positive predictive value, NPV—negative predictive value, LR+—positive likelihood ratio, LR−—negative likelihood ratio.

The biomarkers were also analysed for their diagnostic performance in various combinations. Panels of two-five biomarkers showed even better characteristics, which in the most cases exceeded the respective values of the individual assays. More precisely the particular biomarker panels showed increased diagnostic sensitivity, accuracy and considerably higher NPV as well as positive likelihood ratio (Table 7).

TABLE 7
The diagnostic test performance characteristics of the selected methylation biomarker combinations.

| | | | | | | | | | |
|---|---|---|---|---|---|---|---|---|---|
| | Sensitivity, | Specificity, | Accuracy, | | | | | | Younden |
| VARIABLE | % | % | % | PPV, % | NPV, % | LR+ | LR− | AUC | index |

Combination of genes

| | | | | | | | | | |
|---|---|---|---|---|---|---|---|---|---|
| ZNF677, TAC1 | 69.11 | 97.78 | 77.71 | 98.64 | 57.56 | 31.10 | 0.32 | 0.83 | 0.669 |
| FBN2, TFAP2B | 64.23 | 97.78 | 74.29 | 98.54 | 53.95 | 28.90 | 0.37 | 0.81 | 0.620 |
| FBN2, TAC1 | 70.73 | 97.78 | 78.85 | 98.67 | 58.88 | 31.83 | 0.30 | 0.84 | 0.685 |
| FBN2, SFRP1 | 65.85 | 100.00 | 76.10 | 100.00 | 55.66 | — | 0.34 | 0.83 | 0.659 |
| PCDH8, TAC1 | 67.48 | 97.78 | 76.57 | 98.61 | 56.31 | 30.37 | 0.33 | 0.83 | 0.653 |
| TFAP2B, SFRP1 | 64.23 | 97.78 | 74.29 | 98.54 | 53.95 | 28.90 | 0.37 | 0.81 | 0.620 |
| TAC1, SFRP1 | 69.11 | 97.78 | 77.71 | 98.64 | 57.56 | 31.10 | 0.32 | 0.83 | 0.669 |
| TAC1, ADAMTS19 | 65.04 | 97.78 | 74.86 | 98.56 | 54.52 | 29.27 | 0.36 | 0.81 | 0.628 |
| FBN2, TAC1, SFRP1 | 77.24 | 97.78 | 83.40 | 98.78 | 64.80 | 34.76 | 0.23 | 0.88 | 0.750 |
| ZNF677, FBN2, TAC1 | 76.42 | 97.78 | 82.83 | 98.77 | 63.99 | 34.39 | 0.24 | 0.87 | 0.742 |
| ZNF677, PCDH8, TAC1 | 73.98 | 97.78 | 81.12 | 98.73 | 61.70 | 33.29 | 0.27 | 0.86 | 0.718 |
| FBN2, PCDH8, TAC1 | 73.17 | 97.78 | 80.55 | 98.72 | 60.97 | 32.93 | 0.27 | 0.86 | 0.709 |
| FBN2, TFAP2B, SFRP1 | 73.98 | 97.78 | 81.12 | 98.73 | 61.70 | 33.29 | 0.27 | 0.86 | 0.718 |
| FBN2, PCDH8, SFRP1 | 71.55 | 100.00 | 80.08 | 100.00 | 60.10 | — | 0.29 | 0.86 | 0.715 |
| FBN2, ZNF677, SFRP1 | 73.17 | 100.00 | 81.22 | 100.00 | 61.50 | — | 0.27 | 0.87 | 0.732 |
| TAC1, PCDH8, SFRP1 | 73.98 | 97.78 | 81.12 | 98.73 | 61.70 | 33.29 | 0.27 | 0.86 | 0.718 |
| TAC1, ZNF677, SFRP1 | 76.42 | 97.78 | 82.83 | 98.77 | 63.99 | 34.39 | 0.24 | 0.87 | 0.742 |
| TFAP2B, ZNF677, SFRP1 | 73.17 | 97.78 | 80.55 | 98.72 | 60.97 | 32.93 | 0.27 | 0.86 | 0.709 |
| FBN2, TAC1, SFRP1, ZNF677 | 82.11 | 97.78 | 86.81 | 98.85 | 70.09 | 36.95 | 0.18 | 0.90 | 0.799 |
| FBN2, TAC1, SFRP1, PCDH8 | 78.86 | 97.78 | 84.54 | 98.81 | 66.47 | 35.49 | 0.22 | 0.88 | 0.766 |
| TAC1, PCDH8, SFRP1, ZNF677 | 78.86 | 97.78 | 84.54 | 98.81 | 66.47 | 35.49 | 0.22 | 0.89 | 0.766 |
| ZNF677, FBN2, PCDH8, TAC1 | 78.86 | 97.78 | 84.54 | 98.81 | 66.47 | 35.49 | 0.22 | 0.88 | 0.766 |
| FBN2, TAC1, SFRP1, ZNF677, PCDH8 | 83.74 | 97.78 | 87.95 | 98.88 | 72.05 | 37.68 | 0.17 | 0.91 | 0.815 |

PPV—positive predictive value, NPV—negative predictive value, LR+—positive likelihood ratio, LR−—negative likelihood ratio.

In the group of renal cancer patients, suspected of having clear cell renal cell carcinoma and considered for the surgical tumour resection the presence of the biomarker methylation was associated with 90.4% or even higher probability of having ccRCC, when tested in renal tissues indicating the utility of such assays for more accurate diagnostics in a high-risk population (Table 8).

TABLE 8
The post-test probability estimates for diagnosing ccRCC in an individual when
analysing the particular biomarkers or their selected combinations in patients,
suspected of having renal cancer and undergoing surgical resection. For the
Pre-test probability, the most cited disease prevalences was selected.

| | Post-test probability, % | | |
|---|---|---|---|
| | Whent pre-test | Whent pre-test | Whent pre-test |
| | probability is | probability is | probability is |
| BIOMARKERS | 65% | 70% | 75% |
| ZNF677 | >99.99 | >99.99 | >99.99 |
| FBN2 | >99.99 | >99.99 | >99.99 |
| PCDH8 | >99.99 | >99.99 | >99.99 |
| TFAP2B | 97.25 | 97.80 | 98.28 |
| TAC1 | 98.05 | 98.44 | 98.78 |
| FLRT2 | 88.20 | 90.37 | 92.35 |
| SFRP1 | >99.99 | >99.99 | >99.99 |
| ADAMTS19 | >99.99 | >99.99 | >99.99 |
| BMP7 | >99.99 | >99.99 | >99.99 |
| SIM1 | >99.99 | >99.99 | >99.99 |
| ZNF677, TAC1 | 98.30 | 98.64 | 98.94 |
| FBN2, TFAP2B | 98.17 | 98.54 | 98.86 |
| FBN2, TAC1 | 98.34 | 98.67 | 98.96 |
| FBN2, SFRP1 | >99.99 | >99.99 | >99.99 |
| PCDH8, TAC1 | 98.26 | 98.61 | 98.91 |
| TFAP2B, SFRP1 | 98.17 | 98.54 | 98.86 |
| TAC1, SFRP1 | 98.30 | 98.64 | 98.94 |
| TAC1, ADAMTS19 | 98.19 | 98.56 | 98.87 |
| FBN2, TAC1, SFRP1 | 98.47 | 99.05 | 99.05 |
| ZNF677, FBN2, TAC1 | 98.46 | 99.04 | 99.04 |
| ZNF677, PCDH8, TAC1 | 98.41 | 99.01 | 99.01 |
| FBN2, PCDH8, TAC1 | 98.39 | 99.00 | 99.00 |
| FBN2, TFAP2B, SFRP1 | 98.41 | 99.01 | 99.01 |
| FBN2, PCDH8, SFRP1 | >99.99 | >99.99 | >99.99 |
| FBN2, ZNF677, SFRP1 | >99.99 | >99.99 | >99.99 |
| TAC1, PCDH8, SFRP1 | 98.41 | 99.01 | 99.01 |
| TAC1, ZNF677, SFRP1 | 98.46 | 99.04 | 99.04 |
| TFAP2B, ZNF677, SFRP1 | 98.39 | 99.00 | 99.00 |
| FBN2, TAC1, SFRP1, ZNF677 | 98.56 | 98.85 | 99.11 |
| FBN2, TAC1, SFRP1, PCDH8 | 98.51 | 98.80 | 99.07 |
| TAC1, PCDH8, SFRP1, ZNF677 | 98.51 | 98.81 | 99.07 |
| ZNF677, FBN2, PCDH8, TAC1 | 98.51 | 98.81 | 99.07 |
| FBN2, TAC1, SFRP1, ZNF677, PCDH8 | 98.59 | 98.88 | 99.12 |

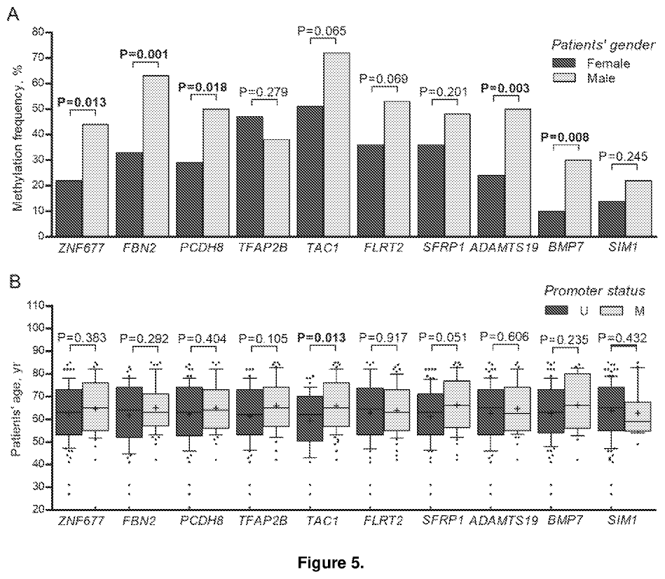

Methylation differences of selected genes was compared with demographic parameters, such as patients' gender and age and obtained results are provided in Table 8 as well as in FIG. 5. Higher methylation frequency of all investigated genes, except of TFAP2B, was observed in males, compared to females, and for ZNF677, FBN2, PCDH8, ADAMTS19 and BMP7 this tendency was statistically significant (P<0.050; FIG. 5A). Besides, methylated status of TAC1 was related to the older patients (P=0.013; FIG. 5B).

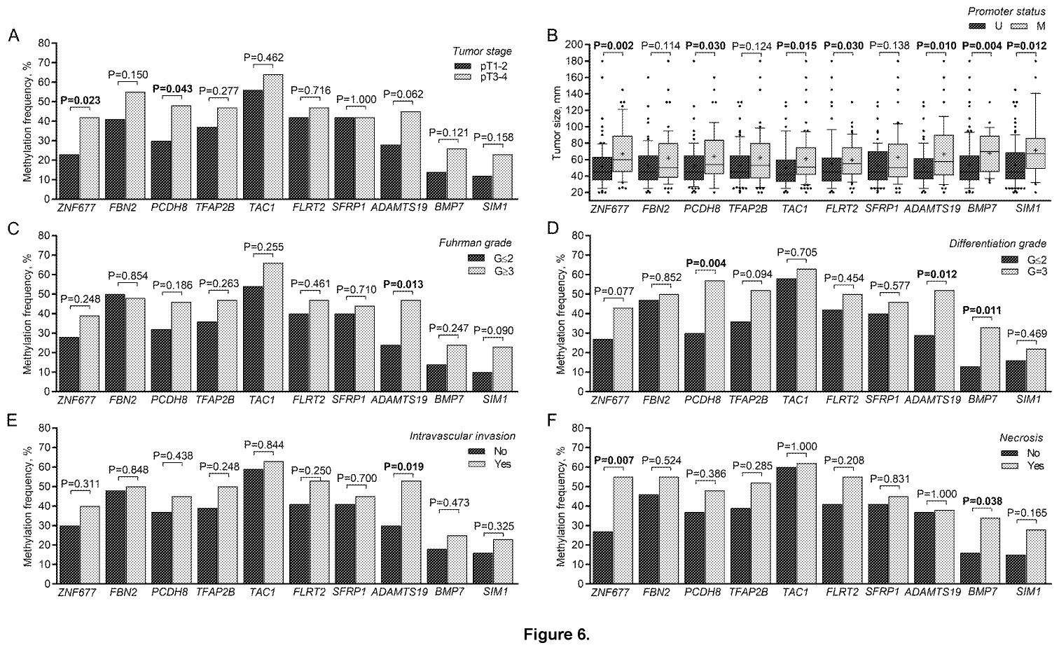

Aberrant methylation of the genes was further analysed according to clinical-pathological patients' characteristics (Table 9 and FIG. 6). Methylation frequencies of all of the genes, except of SFRP1, showed an increasing tendency according to the tumour stage, however the observed association was statistically significant only for ZNF677 and PCDH8 (P=0.023 and P=0.043 respectively; FIG. 6A). Furthermore methylated status of ZNF677, PCDH8, TAC1, FLRT2, ADAMTS19, BMP7 and SIM1 was statistically significant associated with larger tumours (P<0.050; FIG. 6B). Moreover, almost all genes, except of FBN2, were more commonly methylated in tumours with higher Fuhrman grade, but only for ADAMTS19 this association was statistically significant (P=0.013; FIG. 6C) as in the case of tumour intravascular invasion (P=0.019; FIG. 6D). Methylation frequency of all genes was also elevated in higher differentiation grade tumours and for PCDH8, ADAMTS19 and BMP7 this tendency was significant (P=0.004, P=0.012 and P=0.011 respectively; FIG. 6E). In addition, frequent methylation of ZNF677 and BMP7 was related to the presence of tumour necrosis (P=0.007 and P=0.038 respectively; FIG. 6F).

TABLE 9
Associations of promoter methylation with demografic and clinical-
pathological variables of the analysed renal tumour samples.

| | Histology: | | Gender: | | Age (dich.): | |
|---|---|---|---|---|---|---|
| | ccRCC vs. NRT | | Male vs. Female | | >63 yr. vs. ≤63 yr. | |
| VARIABLE | p-value | Frequency, % | p-value | Frequency, % | p-value | Frequency, % |
| ZNF677 | <0.0001 | 33.3 vs. 0.0 | 0.0131 | 43.8 vs. 22.0 | 0.8497 | 34.4 vs. 32.3 |
| FBN2 | <0.0001 | 48.4 vs. 0.0 | 0.0012 | 62.5 vs. 32.8 | 0.7214 | 46.7 vs. 50.0 |
| PCDH8 | <0.0001 | 39.8 vs. 0.0 | 0.0180 | 50.0 vs. 28.8 | 0.8549 | 41.0 vs. 38.7 |
| TFAP2B | <0.0001 | 42.3 vs. 2.2 | 0.2792 | 37.5 vs. 47.5 | 0.1461 | 49.2 vs. 35.5 |
| TAC1 | <0.0001 | 60.2 vs. 2.2 | 0.0648 | 71.9 vs. 50.8 | 0.2703 | 65.6 vs. 54.8 |
| FLRT2 | <0.0001 | 44.7 vs. 11.1 | 0.0693 | 53.1 vs. 35.6 | 0.7179 | 42.6 vs. 46.8 |
| SFRP1 | <0.0001 | 42.3 vs. 0.0 | 0.2009 | 48.4 vs. 35.6 | 0.4679 | 45.9 vs. 38.7 |
| ADAMTS19 | <0.0001 | 37.4 vs. 0.0 | 0.0030 | 50.0 vs. 23.7 | 0.3527 | 32.8 vs. 41.9 |
| BMP7 | 0.0003 | 20.3 vs. 0.0 | 0.0077 | 29.7 vs. 10.2 | 0.5087 | 23.0 vs. 17.7 |
| SIM1 | 0.0079 | 18.0 vs. 0.0 | 0.2454 | 22.2 vs. 13.6 | 0.2387 | 13.0 vs. 23.0 |
| | Age (cont.): | | Tumor stage: | | Tumor size (dich.): | |
| | M vs. U | | pT3-4 vs. pT1-2 | | >45 mm vs. ≤45 mm | |
| VARIABLE | p-value | Mean age, yr. | p-value | Frequency, % | p-value | Frequency, % |
| ZNF677 | 0.3830 | 65 vs. 63 | 0.0231 | 42.4 vs. 22.8 | 0.0124 | 45.0 vs. 22.2 |
| FBN2 | 0.2921 | 65 vs. 62 | 0.1502 | 54.5 vs. 41.1 | 0.3650 | 53.3 vs. 43.5 |
| PCDH8 | 0.4038 | 65 vs. 62 | 0.0429 | 48.5 vs. 29.8 | 0.0679 | 48.3 vs. 31.7 |
| TFAP2B | 0.1049 | 66 vs. 61 | 0.2774 | 47.0 vs. 36.8 | 0.0461 | 51.7 vs. 33.3 |
| TAC1 | 0.0131 | 66 vs. 59 | 0.4615 | 63.6 vs. 56.1 | 0.0971 | 68.3 vs. 52.4 |
| FLRT2 | 0.9169 | 64 vs. 63 | 0.7163 | 47.0 vs. 42.1 | 0.0712 | 53.3 vs. 36.5 |
| SFRP1 | 0.0507 | 66 vs. 61 | 1.0000 | 42.4 vs. 42.1 | 0.5874 | 45.0 vs. 39.7 |
| ADAMTS19 | 0.6064 | 65 vs. 63 | 0.0617 | 45.5 vs. 28.1 | 0.0425 | 46.7 vs. 28.6 |
| BMP7 | 0.2346 | 66 vs. 63 | 0.1212 | 25.8 vs. 14.0 | 0.0431 | 28.3 vs. 12.7 |
| SIM1 | 0.4317 | 63 vs. 64 | 0.1582 | 23.1 vs. 12.3 | 0.0185 | 26.7 vs. 9.7 |
| | Tumor size (cont.): | | Fuhrman grade: | | Differentiation grade: | |
| | M vs. U | | G3-4 vs. G1-2 | | G3 vs. G1-2 | |
| VARIABLE | p-value | Mean size, mm | p-value | Frequency, % | p-value | Frequency, % |
| ZNF677 | 0.0021 | 67.1 vs. 51.6 | 0.2480 | 38.6 vs. 28.0 | 0.0770 | 43.5 vs. 27.3 |
| FBN2 | 0.1142 | 61.7 vs. 52.6 | 0.8541 | 47.8 vs. 50.0 | 0.8525 | 50.0 vs. 47.7 |
| PCDH8 | 0.0295 | 63.7 vs. 52.2 | 0.1856 | 45.7 vs. 32.0 | 0.0044 | 56.5 vs. 29.9 |
| TFAP2B | 0.1236 | 62.2 vs. 52.8 | 0.2633 | 47.1 vs. 36.0 | 0.0936 | 52.2 vs. 36.4 |
| TAC1 | 0.0148 | 61.0 vs. 50.4 | 0.2552 | 65.7 vs. 54.0 | 0.7045 | 63.0 vs. 58.4 |
| FLRT2 | 0.0297 | 59.6 vs. 54.5 | 0.4616 | 47.1 vs. 40.0 | 0.4538 | 50.0 vs. 41.6 |
| SFRP1 | 0.1379 | 62.7 vs. 52.4 | 0.7095 | 44.3 vs. 40.0 | 0.5768 | 45.7 vs. 40.3 |
| ADAMTS19 | 0.0098 | 66.8 vs. 50.8 | 0.0128 | 47.1 vs. 24.0 | 0.0122 | 52.2 vs. 28.6 |
| BMP7 | 0.0039 | 67.7 vs. 54.0 | 0.2468 | 24.3 vs. 14.0 | 0.0114 | 32.6 vs. 13.0 |
| SIM1 | 0.0120 | 71.5 vs. 53.6 | 0.0903 | 22.9 vs. 10.2 | 0.4693 | 21.7 vs. 15.8 |
| | Intravascular | | Fat invasion: | | Necrotic zone: | |
| | invasion: Yes vs. No | | Yes vs. No | | Yes vs. No | |
| VARIABLE | p-value | Frequency, % | p-value | Frequency, % | p-value | Frequency, % |
| ZNF677 | 0.3108 | 40.0 vs. 30.1 | 0.1194 | 42.0 vs. 27.4 | 0.0066 | 55.2 vs. 26.6 |
| FBN2 | 0.8484 | 50.0 vs. 47.7 | 0.3582 | 54.0 vs. 44.4 | 0.5237 | 55.2 vs. 46.2 |
| PCDH8 | 0.4377 | 45.0 vs. 37.3 | 0.7110 | 42.0 vs. 38.4 | 0.3857 | 48.3 vs. 37.2 |
| TFAP2B | 0.2478 | 50.0 vs. 38.6 | 1.0000 | 42.0 vs. 42.5 | 0.2847 | 51.7 vs. 39.4 |
| TAC1 | 0.8445 | 62.5 vs. 59.0 | 0.7110 | 58.0 vs. 61.6 | 1.0000 | 62.1 vs. 59.6 |
| FLRT2 | 0.2503 | 52.5 vs. 41.0 | 0.8548 | 46.0 vs. 43.8 | 0.2079 | 55.2 vs. 41.5 |
| SFRP1 | 0.7003 | 45.0 vs. 41.0 | 1.0000 | 42.0 vs. 42.5 | 0.8308 | 44.8 vs. 41.5 |
| ADAMTS19 | 0.0185 | 52.5 vs. 30.1 | 0.4492 | 42.0 vs. 34.2 | 1.0000 | 37.9 vs. 37.2 |
| BMP7 | 0.4734 | 25.0 vs. 18.1 | 0.8202 | 22.0 vs. 19.2 | 0.0376 | 34.5 vs. 16.0 |
| SIM1 | 0.3246 | 23.1 vs. 15.7 | 0.6409 | 20.0 vs. 16.4 | 0.1654 | 27.6 vs. 15.1 |

Mediana value was used for the dividing patients age and tumour size into two groups. Abbreviations: ccRCC—clear cell renal cell carcinoma; NRT—non-cancerous tumour tissues; M—methylated promoter status; U—unmethylated promoter status; dich.—dichotomous variable; cont.—continuous variable; yr.—years.

### Overall Survival Analysis

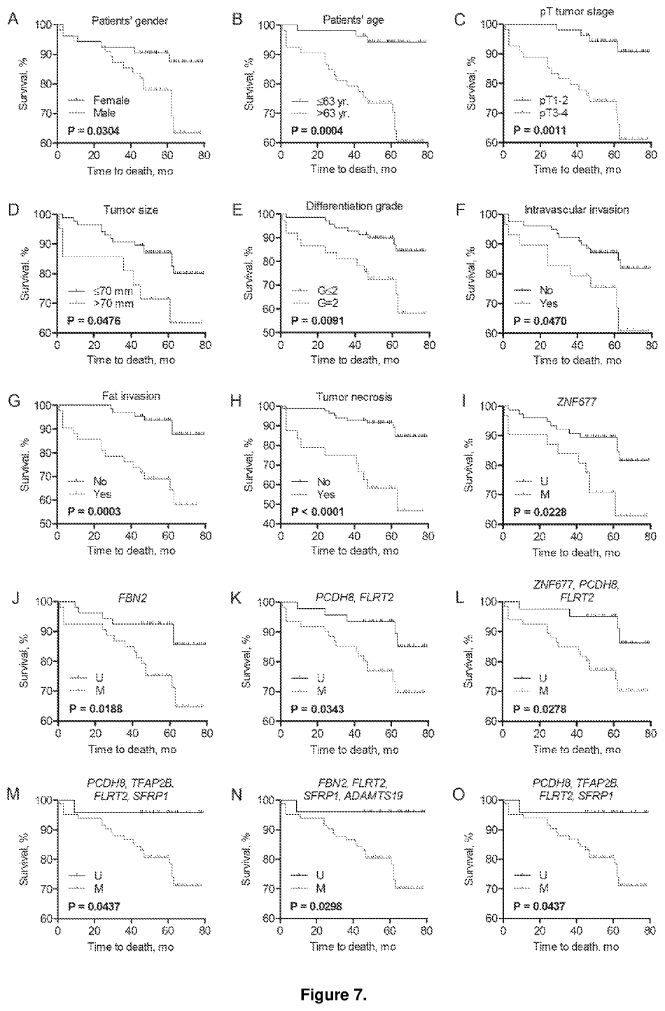

To investigate the performance of the genes for predicting progression of ccRCC, overall survival analysis by comparing Kaplan-Meier curves was performed. The analysis showed significantly lower overall survival rates in ccRCC cases with methylated status of ZNF677 and FBN2 (P=0.0228 and P=0.0188 respectively), in addition to the male and older age patients, as well as higher tumour stage, size, differentiation grade, intravascular or fat invasion and tumour necrosis (all P<0.0500; FIG. 7A-J). Although no associations were observed for other single biomarkers (P>0.0500; data not shown), however, various combinations of genes in which methylation of at least one gene was found, also was significantly associated with poorer overall survival of ccRCC patients (P<0.0500; FIG. 7K-O).

The significance of selected biomarkers methylation as an independent prognostic factor was also supported by univariate and multivariate Cox proportional hazard analyses (Tables 10). In the univariate analysis, methylation of ZNF677, FBN2 and various combinations of two-four biomarkers (when methylated at least one gene) as well as demographic and clinical variables, such as patients' age, gender, tumour stage, differentiation grade, fat invasion and necrosis were significant predictors of the overall survival of ccRCC patients (all P<0.0500; Table 10). In the multivariate Cox regression model, backward entering of covariates revealed significant predictive value of patients' age, tumour stage and necrosis, as well as methylation of at least one of the three genes-ZNF677, PCDH8 and FLRT2 (Table 10). Altogether, this indicates the potential to develop a molecular test for predicting ccRCC progression based solely on DNA methylation biomarkers.

TABLE 10
Univariate and multivariate Cox proportional hazard analysis of the gene methylation biomarkers
and demograpfic as well as and clinical-pathological parameters of ccRCC patients.

| UNIVARIATE ANALYSIS | | | MULTIVARIATE ANALYSIS | | | |
|---|---|---|---|---|---|---|
| | | Hazard ratio | | Hazard ratio | | Hazard ratio |
| Variables | P-value | [95% CI] | P-value | [95% CI] | P-value | [95% CI] |
| Single biomarker | | | | | Biomarkers + DCP parameters | |
| ZNF677 | 0.0288 | 2.61 [1.10-6.17] | backward eliminated | | | |
| FBN2 | 0.0255 | 2.96 [1.14-7.66] | 0.0255 | 2.96 [1.15-7.62] | backward eliminated | |
| Combination of biomarkers | | | | | | |
| ZNF677, FBN2 | 0.0473 | 2.78 [1.02-7.58] | backward eliminated | | | |
| ZNF677, SFRP1 | 0.0481 | 2.76 [1.01-7.51] | backward eliminated | | | |
| ZNF677, BMP7 | 0.0075 | 3.28 [1.38-7.78] | 0.0075 | 3.27 [1.38-7.78] | backward eliminated | |
| ZNF677, SIM1 | 0.0257 | 2.69 [1.13-6.37] | backward eliminated | | | |
| PCDH8, FLRT2 | 0.0435 | 2.82 [1.04-7.68] | backward eliminated | | | |
| ZNF677, FBN2, BMP7 | 0.0473 | 2.78 [1.02-7.58] | backward eliminated | | | |
| ZNF677, PCDH8, FLRT2 | 0.0378 | 3.18 [1.07-9.45] | 0.0378 | 3.18 [1.07-9.45] | 0.0363 | 3.30 [1.09-10.03] |
| ZNF677, BMP7, SIM1 | 0.0497 | 2.39 [1.01-5.67] | backward eliminated | | | |
| PCDH8, SFRP1, BMP7 | 0.0416 | 3.11 [1.05-9.18] | backward eliminated | | | |
| ZNF677, PCDH8, FLRT2, BMP7 | 0.0436 | 3.08 [1.04-9.15] | 0.0436 | 3.08 [1.04-9.15] | backward eliminated | |
| ZNF677, PCDH8, FLRT2, SIM1 | 0.0464 | 3.03 [1.02-9.00] | backward eliminated | | | |
| Demographic and clinicopathological (DCP) parameters | | | | | | |
| Age (>63 yr. vs. ≤63 yr.) | 0.0022 | 6.73 [1.98-22.87] | 0.0027 | 6.61 [1.94-22.58] | 0.0055 | 5.66 [1.67-19.16] |
| Gender (male vs. female) | 0.0382 | 2.73 [1.06-7.08] | 0.0441 | 2.70 [1.03-7.07] | backward eliminated | |
| Stage (pT3-4 vs. pT1-2) | 0.0034 | 5.12 [1.72-15.24] | 0.0477 | 3.13 [1.02-9.64] | 0.0256 | 3.53 [1.17-10.60] |
| Dif. grade (G3 vs. G1-2) | 0.0119 | 3.04 [1.28-7.21] | backward eliminated | | | |
| Fat invasion (Yes vs. No) | 0.0011 | 4.83 [1.87-12.48] | backward eliminated | | | |
| Necrosis (Yes vs. No) | 0.0003 | 4.97 [2.10-11.76] | 0.0281 | 2.73 [1.12-6.66] | 0.0041 | 3.68 [1.52-8.92] |

Abbreviations: CI—confidence interval; yr.—years.

### Transcriptional Expression Analysis of the Selected Target Genes

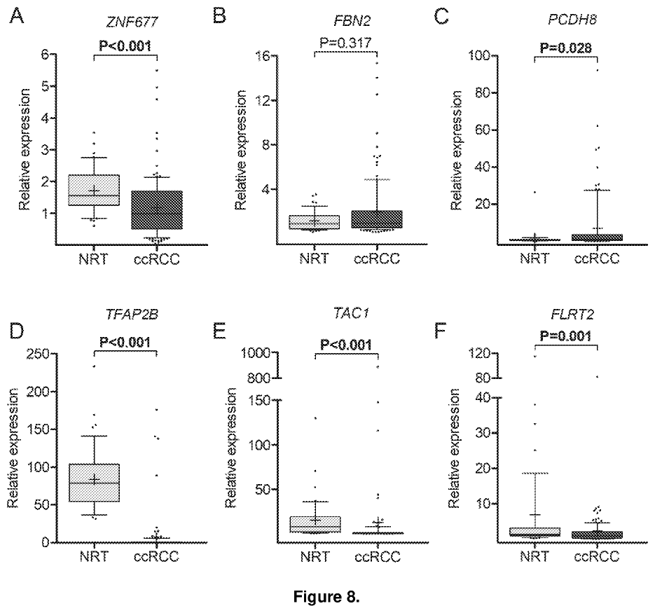

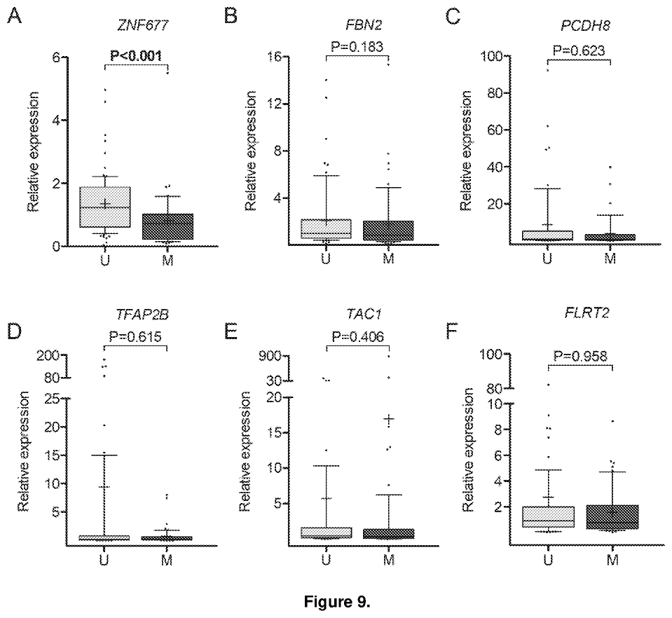

According to the methylation frequencies and with regard to the associations with clinical-pathological variables as well as multivariate Cox proportional hazard analysis the genes ZNF677, FBN2, PCDH8, TFAP2B, TAC1 and FLRT2 were further submitted to the transcriptional expression analysis at mRNA level. Sufficient quantity and quality of RNA was available for 120 ccRCC and 45 NRT samples (Table 1). Transcriptional expression of ZNF677, FBN2 and FLRT2 genes was detected in all ccRCC and NRT samples, while mRNA of TFAP2B, TAC1 and PCDH8 was observed in 118, 112 and 91 ccRCC as well as 45, 45 and 26 NRT samples respectively. Expression levels of ZNF677, TFAP2B, TAC1 and FLRT2 were significantly lower in ccRCC tissues as compared to NRT samples (all P<0.0500). In the case of FBN2 differences of mRNA level were not statistically significant meanwhile significantlly higher expression of PCDH8 was found in ccRCC as compared to NRT (FIG. 8. A-F). Furthermore, lower expression levels of ZNF677 in ccRCC tissues was significantlly associated with methylated promoter status (P=0.0001) proving DNA methylation as a regulatory mechanism responsible for the altered gene expression, while no such correlation was observed for the other selected genes (P>0.0500; FIG. 9. A-F).

Decreasing transcriptional expression level of ZNF677 was signficantlly corelated with various clinical-pathological parameters, including higher tumour stage, Fuhrman and differentiation grade, larger (>45 mm) tumour size, presence of tumour vascular and fat invasions as well as necrosis (all P<0.0500). Lower expression level of FLRT2 was related with presence of tumour necrosis as well (P=0.0062), while decreased expression of TAC1 was observed in larger tumours (P=0.0352). On the contrary, higher mRNA level of FBN2 was significantly associated with larger tumour size and tumour necrosis (P=0.0075 and P=0.0011 respectively) (Table 11).

TABLE 11
Associations of transcriptional expression of selected genes and clinical-
pathological variables of the analysed renal tumour samples.

| | Histology: | | Gender: | | Age (dich.): | | | | Tumor stage: | | Tumor size (dich.): | |
|---|---|---|---|---|---|---|---|---|---|---|---|---|
| | ccRCC vs. NRT | | Male vs. Female | | >63 yr. vs. ≤63 yr. | | Age (cont.) | | pT3-4 vs. PT1-2 | | >45 mm vs. ≤45 mm | |
| VARIABLE | p-value | FC | p-value | FC | p-value | FC | p-value | Rs | p-value | FC | p-value | FC |
| ZNF677 | <0.0001 | −1.9 | 0.3762 | −1.2 | 0.0342 | −1.3 | 0.0040 | −0.26 | 0.0086 | −1.6 | 0.0019 | −1.8 |
| FBN2 | 0.3170 | 1.3 | 0.3261 | 1.1 | 0.5067 | 1.2 | 0.2099 | 0.12 | 0.3240 | 1.3 | 0.0075 | 1.7 |
| PCDH8 | 0.0281 | 2.2 | 0.8551 | 1.1 | 0.8149 | 1.1 | 0.7576 | 0.03 | 0.5356 | 1.3 | 0.2748 | −1.5 |
| TFAP2B | <0.0001 | −270.3 | 0.2287 | 1.5 | 0.9356 | 1.1 | 0.2299 | −0.11 | 0.5587 | 1.1 | 0.4920 | −1.7 |
| TAC1 | <0.0001 | −13.6 | 0.2801 | 1.7 | 0.4617 | −1.6 | 0.3802 | −0.08 | 0.7577 | −1.2 | 0.0352 | −2.2 |
| FLRT2 | 0.0013 | −2.4 | 0.3540 | 1.4 | 0.4983 | −1.1 | 0.1333 | −0.14 | 0.1041 | −1.5 | 0.0872 | −1.5 |

| | Tumor size | | Fuhrman grade: | | Differentiation | | Vascular invasion: | | Fat invasion: | | Necrotic zone: | |
|---|---|---|---|---|---|---|---|---|---|---|---|---|
| | (cont.) | | G3-4 vs. G1-2 | | grade: G3 vs. G1-2 | | Yes vs. No | | Yes vs. No | | Yes vs. No | |
| VARIABLE | p-value | Rs | p-value | FC | p-value | FC | p-value | FC | p-value | FC | p-value | FC |
| ZNF677 | 0.0012 | −0.29 | 0.0056 | −1.6 | 0.0002 | −1.8 | 0.0051 | −1.6 | 0.0211 | −1.6 | 0.0003 | −2.0 |
| FBN2 | 0.0126 | 0.23 | 0.2752 | 1.3 | 0.0199 | 1.6 | 0.7307 | −1.0 | 0.1098 | 1.4 | 0.0011 | 2.2 |
| PCDH8 | 0.2369 | −0.11 | 0.8690 | 1.0 | 0.8405 | 1.2 | 0.5906 | 1.6 | 0.3002 | 1.5 | 0.8529 | 1.2 |
| TFAP2B | 0.6529 | −0.04 | 0.7767 | 1.0 | 0.2229 | −1.6 | 0.2060 | 1.5 | 0.6332 | 1.2 | 0.2937 | −1.7 |
| TAC1 | 0.3201 | −0.09 | 0.6633 | 1.5 | 0.6784 | 1.3 | 0.1886 | −1.9 | 0.8193 | −1.0 | 0.2199 | −1.7 |
| FLRT2 | 0.2278 | −0.11 | 0.1914 | −1.4 | 0.3625 | −1.3 | 0.9035 | 1.1 | 0.0634 | −1.4 | 0.0062 | −2.2 |

Abbreviations: FC—fold change; yr.—years; Rs—Spearman's correlation coefficient.

### DNA Methylation Analysis in Urine

In urine samples, DNA methylation analysis of six biomarkers-ZNF677, FBN2, PCDH8, TFAP2B, TAC1 and FLRT2 was performed by the QMSP method, using the primers and probes provided in Table 4, and was evaluated both quantitatively and qualitatively. For the quantitative evaluation of the particular biomarker, the methylation levels determined according to Formula 1 were used. The qualitative interpretation of the results was made by applying the threshold, calculated according the gene-specific average methylation level in the ASC group. The particular gene was considered as having methylation status when its methylation level was above the particular threshold (Table 12).

TABLE 12
The thresholds applied for the qualitative interpretation
of the gene methylation levels.

| Methylation | Treshold value, % | | | | | |
|---|---|---|---|---|---|---|
| status | ZNF677 | FBN2 | PCDH8 | TFAP2B | TAC1 | FLRT2 |
| M | >0.0171 | >0.0109 | >0.0727 | >0.2924 | >0.1613 | >0.1233 |
| U | ≤0.0171 | ≤0.0109 | ≤0.0727 | ≤0.2924 | ≤0.1613 | ≤0.1233 |

Abbreviations: M/U—methylated/unmethylated status.

Biomarker Performance in Urine for Non-Invasive Diagnostics of ccRCC

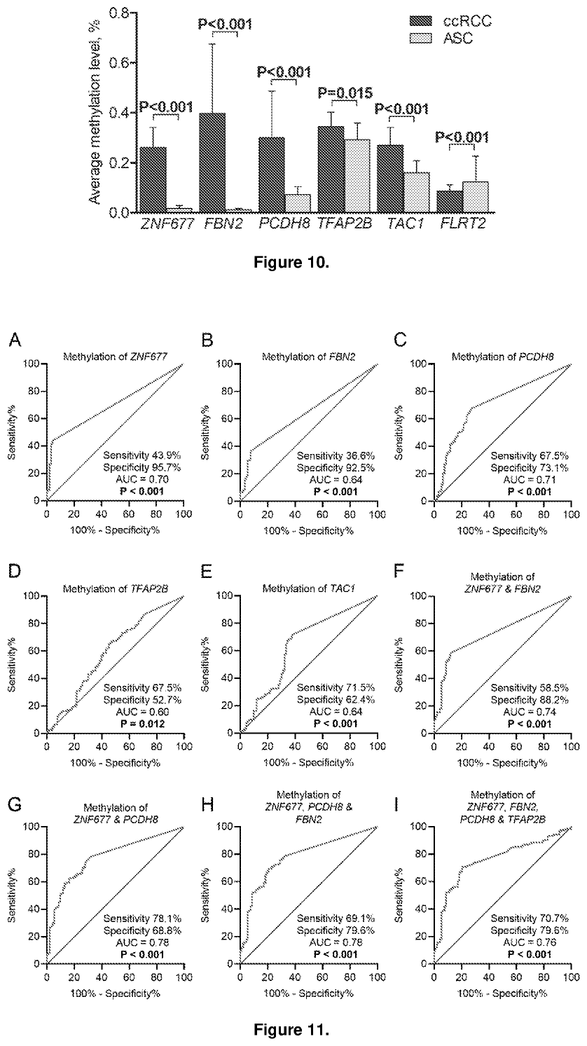

DNA methylation of the selected genes was evaluated in voided urine samples collected from the ccRCC patients (N=123) and asimptomatic controls (N=93). Average methylation levels of all genes, except of FLRT2, were significantly higher in urine of ccRCC cases as compared to ASC group (P<0.0500; FIG. 10). ROC curve analysis revealed moderate-to-high sensitivity and specificity values of single biomarker (all P<0.0001; FIG. 11A-E, Table 13) for the diagnosis of ccRCC. The combination of two-four biomarkers showed even better characteristics (FIG. 11F-I; Table 13) and the best test performance was observed combining only two biomarkers, i.e. ZNF677 and PCDH8 (AUC=0.779, P<0.0001; FIG. 11G, Table 13).

TABLE 13
The test performance characteristics for diagnosing ccRCC when methylation
is analysed in urine of patients diagnosed with ccRCC.

| | | | | | |
|---|---|---|---|---|---|
| | | | | | Youden |
| BIOMARKER | AUC | Specificity, % | Sensitivity, % | P-value | Index |

Single biomarker

| | | | | | |
|---|---|---|---|---|---|
| ZNF677 | 0.698 | 95.70 | 43.90 | <0.0001 | 0.396 |
| FBN2 | 0.644 | 92.47 | 36.59 | <0.0001 | 0.291 |
| PCDH8 | 0.711 | 73.12 | 67.48 | <0.0001 | 0.406 |
| TFAP2B | 0.596 | 52.96 | 67.48 | 0.0148 | 0.202 |
| TAC1 | 0.644 | 62.37 | 71.54 | 0.0001 | 0.339 |

Combination of biomarkers

| | | | | | |
|---|---|---|---|---|---|
| ZNF677 + FBN2 | 0.737 | 88.17 | 58.554 | <0.0001 | 0.467 |
| ZNF677 + PCDH8 | 0.779 | 68.82 | 78.05 | <0.0001 | 0.469 |
| ZNF677 + TFAP2B | 0.719 | 95.70 | 42.28 | <0.0001 | 0.380 |
| ZNF677 + TAC1 | 0.734 | 62.37 | 77.24 | <0.0001 | 0.396 |
| FBN2 + PCDH8 | 0.730 | 72.04 | 70.73 | <0.0001 | 0.428 |
| ZNF677 + FBN2 + PCDH8 | 0.779 | 79.57 | 69.11 | <0.0001 | 0.487 |
| ZNF677 + FBN2 + TFAP2B | 0.727 | 88.17 | 58.54 | <0.0001 | 0.467 |
| ZNF677 + FBN2 + TAC1 | 0.755 | 81.72 | 62.60 | <0.0001 | 0.443 |
| ZNF677 + PCDH8 + TFAP2B | 0.758 | 82.80 | 61.79 | <0.0001 | 0.446 |
| ZNF677 + PCDH8 + TAC1 | 0.743 | 61.29 | 78.86 | <0.0001 | 0.402 |
| ZNF677 + TFAP2B + TAC1 | 0.722 | 84.95 | 52.03 | <0.0001 | 0.370 |
| FBN2 + PCDH8 + TFAP2B | 0.711 | 79.57 | 56.91 | <0.0001 | 0.365 |
| FBN2 + PCDH8 + TAC1 | 0.712 | 56.99 | 80.49 | <0.0001 | 0.375 |
| ZNF677 + FBN2 + PCDH8 + TFAP2B | 0.760 | 79.57 | 70.73 | <0.0001 | 0.503 |
| ZNF677 + FBN2 + PCDH8 + TAC1 | 0.754 | 82.80 | 60.98 | <0.0001 | 0.438 |
| ZNF677 + FBN2 + TFAP2B + TAC1 | 0.755 | 81.72 | 62.60 | <0.0001 | 0.443 |
| ZNF677 + PCDH8 + TFAP2B + TAC1 | 0.730 | 53.76 | 82.93 | <0.0001 | 0.367 |
| ZNF677 + FBN2 + PCDH8 + | 0.753 | 82.80 | 60.98 | <0.0001 | 0.438 |
| TFAP2B + TAC1 | | | | | |

AUC—area under the curve.

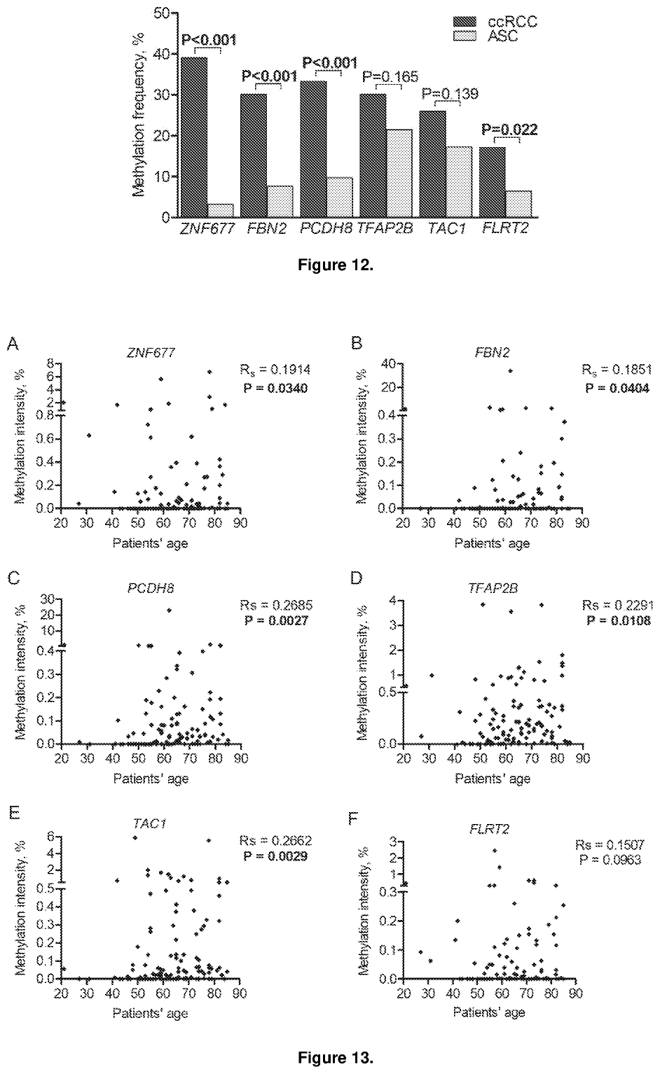

The methylation frequencies determined in urine samples of ccRCC and ASC cases, using the estimated thresholds, are depicted in FIG. 12. Methylation frequencies of all genes were higher in urine from ccRCC patients, compared to ASC and statistically significant difference was obtained for ZNF677, FBN2, PCDH8 and FLRT2 (P<0.0500; FIG. 12). The separate biomarkers and their combinations had considerably high specificity (278.5%) and positive predictive values (276.6%) (Table 14). Moreover, the analysis indicated the potential utility of these biomarkers for the non-invasive diagnostics with the post-test probability for ccRCC detection reaching >95.0% (Table 15).

TABLE 14
The diagnostic test performance characteristics of the biomarkers
and their combinations when analysing the methylation in urine

| | | | | | | | |
|---|---|---|---|---|---|---|---|
| BIOMARKER | Sensitivity, % | Specificity, % | Accuracy, % | PPV, % | NPV, % | LR+ | LR− |

Single biomarker

| | | | | | | | |
|---|---|---|---|---|---|---|---|
| ZNF677 | 39.02 | 96.77 | 56.35 | 98.58 | 40.48 | 12.10 | 0.63 |
| FBN2 | 30.08 | 92.47 | 48.80 | 90.31 | 36.18 | 4.00 | 0.76 |
| PCDH8 | 33.33 | 90.32 | 50.43 | 88.93 | 36.73 | 3.44 | 0.74 |
| TFAP2B | 30.08 | 78.49 | 44.60 | 76.55 | 32.48 | 1.40 | 0.89 |
| TAC1 | 26.02 | 82.80 | 43.05 | 77.92 | 32.41 | 1.51 | 0.89 |
| FLRT2 | 17.07 | 93.55 | 40.01 | 86.06 | 32.59 | 2.65 | 0.89 |

Combination of biomarkers

| | | | | | | | |
|---|---|---|---|---|---|---|---|
| ZNF677, FBN2 | 52.03 | 89.25 | 63.20 | 91.86 | 44.36 | 4.84 | 0.54 |
| ZNF677, PCDH8 | 56.10 | 87.10 | 65.40 | 91.03 | 45.95 | 4.35 | 0.50 |
| ZNF677, FBN2, PCDH8 | 64.23 | 80.65 | 69.16 | 88.56 | 49.14 | 3.32 | 0.44 |

PPV—positive predictive value, NPV—negative predictive value, LR+—positive likelihood ratio, LR−—negative likelihood ratio.

TABLE 15
The post-test probability estimates for diagnosing ccRCC in
an individual when analysing the particular biomarkers in urine
of patients suspected of having ccRCC. For the Pre-test probability,
the most cited disease prevalences was selected.

| | Post-test probability, % | | |
|---|---|---|---|
| | Whent pre-test | Whent pre-test | Whent pre-test |
| | probability is | probability is | probability is |
| BIOMARKERS | 65% | 70% | 75% |

Single biomarker

| | | | |
|---|---|---|---|
| ZNF677 | 95.74 | 96.58 | 97.32 |
| FBN2 | 88.14 | 90.32 | 92.31 |
| PCDH8 | 86.47 | 88.92 | 91.16 |
| TFAP2B | 72.22 | 76.56 | 80.77 |
| TAC1 | 73.71 | 77.89 | 81.91 |
| FLRT2 | 83.11 | 86.08 | 88.83 |

Combination of biomarkers

| | | | |
|---|---|---|---|
| ZNF677, FBN2 | 89.99 | 91.87 | 93.56 |
| ZNF677, PCDH8 | 88.99 | 91.03 | 92.88 |
| ZNF677, FBN2, | 86.04 | 88.57 | 90.88 |
| PCDH8 | | | |

### Prognostic Value of the Biomarkers in Urine of Clear Cell Renal Cell Carcinoma

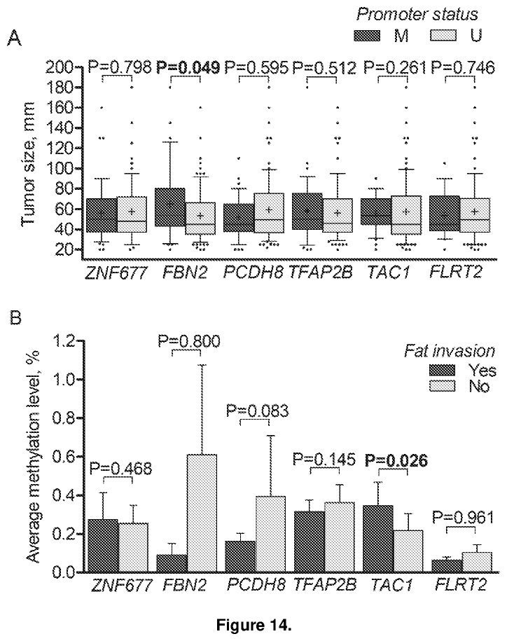

The biomarker methylation in urine was further analysed for the potential to predict ccRCC progression. As was indicated previously, older age of patients, larger tumour size and fat invasion are significantly associated with shorter overall survival of the patients, diagnosed with ccRCC (FIG. 7B, D, G). According to this, methylation intensities of all investigated genes (except of FLRT2) in urine of ccRCC patients were significantly correlated with the patients' age (all P<0.0500; FIG. 13), however no such correlation was observed in the ASC group (data not shown). In addition, methylated status of FBN2 was more common in the case of larger tumour size (P=0.049; FIG. 14A). Moreover, statistically significant higher methylation intensity of TAC1 was established in the urine samples from patients, diagnosed with locally advanced, particularly characterized by the fat invasion, clear cell renal cell carcinoma (P=0.026; FIG. 14B). Meanwhile, after Kaplan-Meier curve analysis, no significant associations between methylated status of selected biomarkers in urine of ccRCC patients and their overall survival were observed (all P>0.0500; data not shown).

DNA Methylation Analysis in Urine of Patients Diagnosed with Small Renal Masses

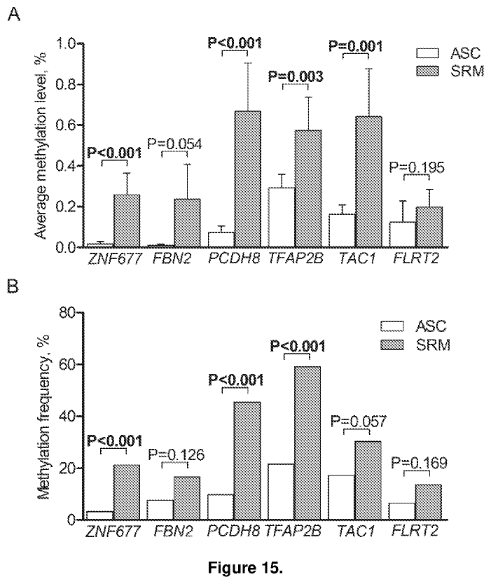

Aiming to evaluate the value of the biomarkers for the early diagnosis, the genes ZNF677, FBN2, PCDH8, TFAP2B, TAC1 and FLRT2 were analysed in voided urine samples (N=66), collected from patients diagnosed with small renal masses (SRM). DNA methylation levels of all genes, except FLRT2, were higher in patients with clinically localized renal masses as compared to asymptomatic controls (FIG. 15A). According to the qualitative analysis, methylation was also more frequent in cases with SRM and for the ZNF677, PCDH8 and TFAP2B this tendency was statistically significant (P<0.050; FIG. 15B). Altogether, this proves that even small renal masses displaying an indolent course contribute significantly to the amount of methylated DNA detectable in urine, therefore, such epigenetic alterations can be used as novel non-invasive biomarkers for the early diagnosis of ccRCC.

Active Surveillance of Patients Diagnosed with Small Renal Masses

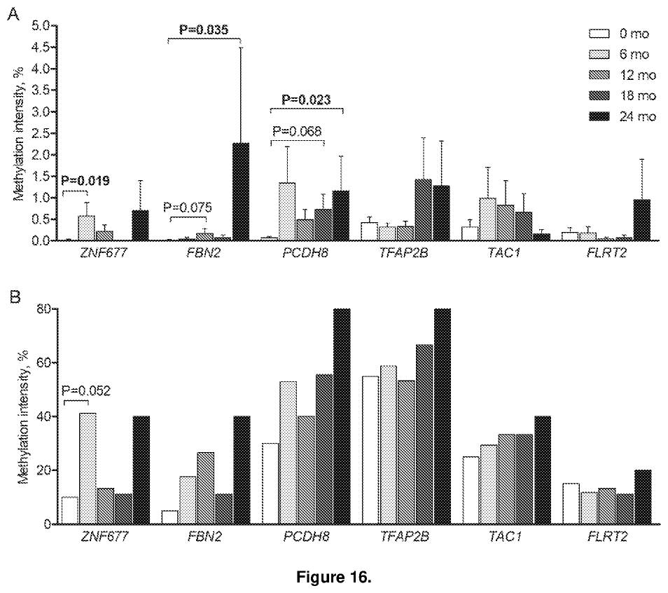

Analysing urine samples collected from patients with SRM at the different time points (0, 6, 12, 18, 24 months) after diagnosis, the tendency of increasing methylation levels was detected in all genes analysed and for ZNF677, FBN2 as well as PCDH8 this increment was significant (P <0.050; FIG. 16A). The similar tendency was observed in the qualitative analysis as well, although the differences in methylation frequencies at the individual time point of active surveillance were not significant (all P>0.050; FIG. 16B). Collectively, obtained results show the possibility to apply some of biomarkers for the active surveillance of patients diagnosed with clinically localized renal masses.

1. A method for identification of ceRCC in patients diagnosed with ccRCC or suspected of having ccRCC consist of:
1. obtainment of biological sample from said individuals;
2. determination of the methylation status of at least one biomarker described herein in the sample DNA, wherein the panel of biomarkers comprise of:
a) DNA sequences defined by ZNF677 (SEQ ID NOs: 1 and/or 11), FBN2 (SEQ ID NOs: 2 and/or 12), PCDH8 (SEQ ID NOs: 3 and/or 13) TFAP2B (SEQ ID NOs: 4 and/or 14), TAC1 (SEQ ID NOs: 5 and/or 15), FLRT2 (SEQ ID NOs: 6 and/or 16), SFRP1 (SEQ ID NO: 7), ADAMTS19 (SEQ ID NO: 8), BMP7 (SEQ ID NO: 9) and SIM1 (SEQ ID NO: 10); or b) DNA sequences, that are complementary to that nucleotide sequences; or
c) at least a fragment of any sequences of a) and/or b).
3) identification of the samples as containing nucleic acids from cells that are cancerous or precursor to cancerous, if DNA methylation is observed in at least one of said biomarkers in test sample; and
4) identification of the individuals, as having ceRCC if DNA methylation is observed in at least one of said biomarkers in the sample.

2. The method according to claim 1, wherein the sample consists of or is suspected to consist of renal tissue, renal cells or a body fluid, preferably urine.

3. The method according to claim 1, which encompass detection of DNA methylation status of at least one of the biomarkers, wherein the presence of methylated biomarker refer to:
a) the presence of ccRCC or the predisposition to ceRCC; or
b) the presence of aggressive or potentially aggressive ccRCC.

4. The method according to claim 1, wherein methylation status in DNA sequence of said biomarkers in the sample is detected by using at least one of the primers or probes indicated by SEQ ID NOs: 18-75.

5. A method for monitoring/active surveillance of individuals diagnosed with SRM, encompassing the steps of:
1. obtainment of biological sample from said individuals;
2. determination of the methylation status of at least one biomarker described herein in the sample DNA, wherein the panel of biomarkers comprise of:
a) nucleotide sequences consisting of ZNF677 (SEQ ID NO: 11), FBN2 (SEQ ID NO: 12), PCDH8 (SEQ ID NO: 11), TFAP2B (SEQ ID NO: 14), TAC1 (SEQ ID NO: 15) and FLRT2 (SEQ ID NO: 16); or
b) DNA sequences, that are complementary to that nucleotide sequences; or
c) at least a fragment of any sequences of a) and/or b).

6. The method according to claim 5, wherein the sample is obtained from an individual having SRM.

7. The method according to claim 5, wherein the sample consists of or is suspected to consist of body fluid, preferably urine.

8. The method according to claim 5, which encompasses detection of DNA methylation level and/or DNA methylation status of at least one of the biomarkers, wherein the presence of methylated biomarker refers to disease progression.

9. The method according to claim 5, wherein methylation level and/or methylation status in DNA sequence of said biomarkers in the sample is detected by using at least one of the primers or probes indicated by SEQ ID NOs: 58-75.

10. Primers for evaluating the DNA methylation status of at least one biomarker, including ZNF677 (SEQ ID NOs: 18-21), FBN2 (SEQ ID NOs: 22-25), PCDH8 (SEQ ID NOs: 26-29), TFAP2B (SEQ ID NOs: 30-33), TAC1 (SEQ ID NOs: 34-37), FLRT2 (SEQ ID NOs: 38-41), SFRP1 (SEQ ID NOs: 42-45), ADAMTS19 (SEQ ID NOs: 46-49), BMP7 (SEQ ID NOs: 50-53) and SIM1 (SEQ ID NOs: 54-57).

11. Primers for evaluating the DNA methylation level of at least one biomarker, including ZNF677 (SEQ ID NOs: 58-59), FBN2 (SEQ ID NOs: 61-62), PCDH8 (SEQ ID NOs: 64-65), TFAP2B (SEQ ID NOs: 67-68), TAC1 (SEQ ID NOs: 70-71) and FLRT2 (SEQ ID NOs: 73-74).

12. Probes for evaluating the DNA methylation level of at least one biomarker, including ZNF677 (SEQ ID NO: 60), FBN2 (SEQ ID NO: 63), PCDH8 (SEQ ID NO: 66), TFAP2B (SEQ ID NO: 69), TAC1 (SEQ ID NO: 72) and FLRT2 (SEQ ID NO: 75).

13. A kit for evaluation the DNA methylation status in a sample comprising renal tissue, renal cells, urine or nucleic acids from renal tissue, renal cells or urine, wherein methylation status of at least one of biomarkers ZNF677 (SEQ ID NOs: 1 and/or 11), FBN2 (SEQ ID NOs: 2 and/or 12), PCDH8 (SEQ ID NOs: 3 and/or 13) TFAP2B (SEQ ID NOs: 4 and/or 14), TAC1 (SEQ ID NOs: 5 and/or 15), FLRT2 (SEQ ID NOs: 6 and/or 16), SFRP1 (SEQ ID NO: 7), ADAMTS19 (SEQ ID NO: 8), BMP7 (SEQ ID NO: 9) and SIM1 (SEQ ID NO: 10) is determined.

14. The kit according to claim 13, wherein the evaluation of DNA methylation status of at least one of said biomarkers comprises qualitative MSP reaction with at least one of amplification primer of biomarkers selected from ZNF677 (SEQ ID NOs: 18-21). FBN2 (SEQ ID NOs: 22-25), PCDH8 (SEQ ID NOs: 26-29), TEAP2B (SEQ ID NOs: 30-33). TAC1 (SEQ ID NOs: 34-37), FLRT2 (SEQ ID NOs: 38-41), SFRP1 (SEQ ID NOs: 42-45), ADAMTS19 (SEQ ID NOS: 46-49), BMP7 (SEQ ID NOs: 50-53) and SIM1 (SEQ ID NOS: 54-57).

15. A kit for evaluation the DNA methylation level in a sample comprising renal tissue, renal cells, urine or nucleic acids from renal tissue, renal cells or urine, wherein methylation level of at least one of biomarkers ZNF677 (SEQ ID NO: 11), FBN2 (SEQ ID NO: 12), PCDH8 (SEQ ID NO: 11), TFAP2B (SEQ ID NO: 14), TAC1 (SEQ ID NO: 15) and FLRT2 (SEQ ID NO: 16) is determined.

16. The kit according to claim 15, wherein the evaluation of DNA methylation level of at least one of said biomarkers comprises quantitative MSP reaction with at least one of amplification primer of biomarkers selected from ZNF677 (SEQ ID NOs: 58-59), FBN2 (SEQ ID NOs: 61-62), PCDH8 (SEQ ID NOs: 64-65), TFAP2B (SEQ ID NOs: 67-68), TAC1 (SEQ ID NOs: 70-71) and FLRT2 (SEQ ID NOs: 73-74).

17. The kit according to claim 16, wherein the kit comprises at least one probes for evaluating the DNA methylation level of at least one biomarker selected from ZNF677 (SEQ ID NO: 60), FBN2 (SEQ ID NO: 63), PCDH8 (SEQ ID NO: 66), TFAP2B (SEQ ID NO: 69), TAC1 (SEQ ID NO: 72) and FLRT2 (SEQ ID NO: 75).


SEQUENCE LISTING (78 sequences)

SEQ ID NO: 1 - DNA, 156 bp, Homo sapiens
tcgcgggttataggtttttacgtttcgttgtcgggttttggggtcgttttgtaggttaaaatttcgaatttgtttatttttttcgcggcgtggttttaagacgtttttagtttcgtcgtttcgagagggtttagagatttgcgtttatttcggatt

SEQ ID NO: 2 - DNA, 182 bp, Homo sapiens
tttaatattcgttttcggagcgtacgggaattcgtcgagttttgcgtgtaggttttttttttttttgaggtttatattttttgaaattttacgttagggtttttgtaattttttttttcgttcgttgacggttttggagtcgttcggggttttaggtcggttatgtaacgtgtatcgttcgg

SEQ ID NO: 3 - DNA, 146 bp, Homo sapiens
tttagagttcgttggaggttcggagttgttattcgtagatttttttcgtatagggttcgtaaagagcgtgatttcgagagtttgagattgacgttcgatttggaaattagagaagatttttttagtttttcggatcgtatttgagg

SEQ ID NO: 4 - DNA, 90 bp, Homo sapiens
ttcgaagattttaagagtgggcgatttataggcgcggtcggtaagtttttgggggattcgggttcggacgagcgtttataggtagcgttt

SEQ ID NO: 5 - DNA, 139 bp, Homo sapiens
ggtattgagtaggcgaaagagcgcgttcggatttttttttcggcggtagttatcgagagtgcggagcgattagcgtgcgttcggaggaattagagaaatttagtatttcgcgggattgttcgtcgtagtaagtgttcgc

SEQ ID NO: 6 - DNA, 277 bp, Homo sapiens
tagtatttggagcgagttttgcgttcgttttcgcgtagcgtcgtacgttcggtttcgagttgttcgtatatacgcgtcggaggagagttcgtttagttttttcgtcgagtttcgggattttttaaattcgaggagtttcggcgtcgcggggtagttttttgtcgtttttttcgttcgttgtattttttttggggttcgttggtttggcgaagcggagagggggaggcggaggaggagagaaggcgggggtcgcggcggtcgaagttaagagaaagtg

SEQ ID NO: 7 - DNA, 156 bp, Homo sapiens
tcgcgtttggttttagtaaatcgaattcgttcgcgagggaggcgattggttttcgcgtcggtgacggacgtggtaacgagtgcggttcgtttcgtcgggagttgattggttgcgcggggcggtttcgagggttcggtcgtaggagtttcgcgtatt

SEQ ID NO: 8 - DNA, 157 bp, Homo sapiens
aaagggtttgggtaaattcgtcgttcggttttttagcgtttcggggaggtcgttgcgtttcggagtggatcgcgttggaggcgtgcgtcgggcgagaagtcgcggtcgcgggagcgtagtatggggaagaatcgcgagatgcgtttgatttatattt

SEQ ID NO: 9 - DNA, 161 bp, Homo sapiens
gttttttaagttttgcggtgcgttcgggcgagtgcggggcgaggggttcggggttagtatcgagtagggggcgggggttcgggtagagcgcggtcggtcggggaggggttatgtttggcgcgggcgtagcggggttcgtttgtagtaagtgatcgagcggc

SEQ ID NO: 10 - DNA, 130 bp, Homo sapiens
gtgaagtagaagacgtttcgcgttcgtttagtagtttcgtagtttcgcggtggtgtgggagaggtcgcggcgttttttattttcgggggagtttgcgaggggttgtcgcgagcgcgttatttgttaattt

SEQ ID NO: 11 - DNA, 96 bp, Homo sapiens
ggcgttttcgggtgagttttcgtttttcgggtttaagtttgcgtttcgcgggttataggtttttacgtttcgttgtcgggttttggggtcgttttg

SEQ ID NO: 12 - DNA, 102 bp, Homo sapiens
tgacggttttggagtcgttcggggttttaggtcggttatgtaacgtgtatcgttcggggttgtcggttgtattttcgtcgcgtttcgtcgtttattgcgtta

SEQ ID NO: 13 - DNA, 91 bp, Homo sapiens
tagagtgagggcgggttcgcgcgtttttagagttcgttggaggttcggagttgttattcgtagatttttttcgtatagggttcgtaaagag

SEQ ID NO: 14 - DNA, 118 bp, Homo sapiens
cgggatagtttttgaaagttcggcgtagagtcgtttcgaagattttaagagtgggcgatttataggcgcggtcggtaagtttttgggggattcgggttcggacgagcgtttataggta

SEQ ID NO: 15 - DNA, 107 bp, Homo sapiens
gagcgattagcgtgcgttcggaggaattagagaaatttagtatttcgcgggattgttcgtcgtagtaagtgttcgcgcggtgttggtcgcggttgttcgggttattt

SEQ ID NO: 16 - DNA, 92 bp, Homo sapiens
agtttttagatttacgtcgggcgggcgtagtatttggagcgagttttgcgttcgttttcgcgtagcgtcgtacgttcggtttcgagttgttc

SEQ ID NO: 17 - DNA, 133 bp, Homo sapiens
aaccaataaaacctactcctcccttaaaaattacaaaaaccacaacctaataaaaaaaataaccaccacccaacacacaataacaaacacaaattcacaatccaaaaaacttactaaacctcctccatcacca

SEQ ID NO: 18 - DNA, 21 bp, Artificial Sequence
  Chemically Synthetized
tcgcgggttataggtttttac

SEQ ID NO: 19 - DNA, 23 bp, Artificial Sequence
  Chemically Synthetized
aatccgaaataaacgcaaatctc

SEQ ID NO: 20 - DNA, 25 bp, Artificial Sequence
  Chemically Synthetized
gttttgtgggttataggtttttatg

SEQ ID NO: 21 - DNA, 27 bp, Artificial Sequence
  Chemically Synthetized
tttaatccaaaataaacacaaatctct

SEQ ID NO: 22 - DNA, 22 bp, Artificial Sequence
  Chemically Synthetized
tttaatattcgttttcggagcg

SEQ ID NO: 23 - DNA, 22 bp, Artificial Sequence
  Chemically Synthetized
ccgaacgatacacgttacataa

SEQ ID NO: 24 - DNA, 29 bp, Artificial Sequence
  Chemically Synthetized
gtagttttttaatatttgtttttggagtg

SEQ ID NO: 25 - DNA, 25 bp, Artificial Sequence
  Chemically Synthetized
accccaaacaatacacattacataa

SEQ ID NO: 26 - DNA, 21 bp, Artificial Sequence
  Chemically Synthetized
tttagagttcgttggaggttc

SEQ ID NO: 27 - DNA, 22 bp, Artificial Sequence
  Chemically Synthetized
cctcaaatacgatccgaaaaac

SEQ ID NO: 28 - DNA, 24 bp, Artificial Sequence
  Chemically Synthetized
gtttttagagtttgttggaggttt

SEQ ID NO: 29 - DNA, 25 bp, Artificial Sequence
  Chemically Synthetized
caacctcaaatacaatccaaaaaac

SEQ ID NO: 30 - DNA, 22 bp, Artificial Sequence
  Chemically Synthetized
ttcgaagattttaagagtgggc

SEQ ID NO: 31 - DNA, 22 bp, Artificial Sequence
  Chemically Synthetized
aaacgctacctataaacgctcg

SEQ ID NO: 32 - DNA, 24 bp, Artificial Sequence
  Chemically Synthetized
gttttgaagattttaagagtgggt

SEQ ID NO: 33 - DNA, 24 bp, Artificial Sequence
  Chemically Synthetized
ccaaacactacctataaacactca

SEQ ID NO: 34 - DNA, 22 bp, Artificial Sequence
  Chemically Synthetized
ggtattgagtaggcgaaagagc

SEQ ID NO: 35 - DNA, 22 bp, Artificial Sequence
  Chemically Synthetized
gcgaacacttactacgacgaac

SEQ ID NO: 36 - DNA, 25 bp, Artificial Sequence
  Chemically Synthetized
taaggtattgagtaggtgaaagagt

SEQ ID NO: 37 - DNA, 26 bp, Artificial Sequence
  Chemically Synthetized
cacaaacacttactacaacaaacaat

SEQ ID NO: 38 - DNA, 22 bp, Artificial Sequence
  Chemically Synthetized
tagtatttggagcgagttttgc

SEQ ID NO: 39 - DNA, 22 bp, Artificial Sequence
  Chemically Synthetized
cactttctcttaacttcgaccg

SEQ ID NO: 40 - DNA, 25 bp, Artificial Sequence
  Chemically Synthetized
gtagtatttggagtgagttttgtgt

SEQ ID NO: 41 - DNA, 23 bp, Artificial Sequence
  Chemically Synthetized
ccactttctcttaacttcaacca

SEQ ID NO: 42 - DNA, 22 bp, Artificial Sequence
  Chemically Synthetized
tcgcgtttggttttagtaaatc

SEQ ID NO: 43 - DNA, 21 bp, Artificial Sequence
  Chemically Synthetized
aatacgcgaaactcctacgac

SEQ ID NO: 44 - DNA, 25 bp, Artificial Sequence
  Chemically Synthetized
gagttgtgtttggttttagtaaatt

SEQ ID NO: 45 - DNA, 24 bp, Artificial Sequence
  Chemically Synthetized
aaaatacacaaaactcctacaacc

SEQ ID NO: 46 - DNA, 22 bp, Artificial Sequence
  Chemically Synthetized
aaagggtttgggtaaattcgtc

SEQ ID NO: 47 - DNA, 24 bp, Artificial Sequence
  Chemically Synthetized
aaatataaatcaaacgcatctcgc

SEQ ID NO: 48 - DNA, 24 bp, Artificial Sequence
  Chemically Synthetized
taaagggtttgggtaaatttgttg

SEQ ID NO: 49 - DNA, 26 bp, Artificial Sequence
  Chemically Synthetized
acaaatataaatcaaacacatctcac

SEQ ID NO: 50 - DNA, 22 bp, Artificial Sequence
  Chemically Synthetized
gttttttaagttttgcggtgcg

SEQ ID NO: 51 - DNA, 20 bp, Artificial Sequence
  Chemically Synthetized
gccgctcgatcacttactac

SEQ ID NO: 52 - DNA, 24 bp, Artificial Sequence
  Chemically Synthetized
gttgttttttaagttttgtggtgt

SEQ ID NO: 53 - DNA, 22 bp, Artificial Sequence
  Chemically Synthetized
caccactcaatcacttactaca

SEQ ID NO: 54 - DNA, 21 bp, Artificial Sequence
  Chemically Synthetized
gtgaagtagaagacgtttcgc

SEQ ID NO: 55 - DNA, 22 bp, Artificial Sequence
  Chemically Synthetized
aaattaacaaataacgcgctcg

SEQ ID NO: 56 - DNA, 24 bp, Artificial Sequence
  Chemically Synthetized
taggtgaagtagaagatgttttgt

SEQ ID NO: 57 - DNA, 25 bp, Artificial Sequence
  Chemically Synthetized
ccaaattaacaaataacacactcac

SEQ ID NO: 58 - DNA, 21 bp, Artificial Sequence
  Chemically Synthetized
ggcgttttcgggtgagttttc

SEQ ID NO: 59 - DNA, 20 bp, Artificial Sequence
  Chemically Synthetized
caaaacgaccccaaaacccg

SEQ ID NO: 60 - DNA, 30 bp, Artificial Sequence
  Chemically Synthetized
gaaacgtaaaaacctataacccgcgaaacg

SEQ ID NO: 61 - DNA, 20 bp, Artificial Sequence
  Chemically Synthetized
tgacggttttggagtcgttc

SEQ ID NO: 62 - DNA, 22 bp, Artificial Sequence
  Chemically Synthetized
taacgcaataaacgacgaaacg

SEQ ID NO: 63 - DNA, 27 bp, Artificial Sequence
  Chemically Synthetized
cgacaaccccgaacgatacacgttaca

SEQ ID NO: 64 - DNA, 18 bp, Artificial Sequence
  Chemically Synthetized
tagagtgagggcgggttc

SEQ ID NO: 65 - DNA, 22 bp, Artificial Sequence
  Chemically Synthetized
ctctttacgaaccctatacgaa

SEQ ID NO: 66 - DNA, 28 bp, Artificial Sequence
  Chemically Synthetized
cgaacctccaacgaactctaaaaacgcg

SEQ ID NO: 67 - DNA, 22 bp, Artificial Sequence
  Chemically Synthetized
cgggatagtttttgaaagttcg

SEQ ID NO: 68 - DNA, 20 bp, Artificial Sequence
  Chemically Synthetized
tacctataaacgctcgtccg

SEQ ID NO: 69 - DNA, 30 bp, Artificial Sequence
  Chemically Synthetized
gagtcgtttcgaagattttaagagtgggcg

SEQ ID NO: 70 - DNA, 19 bp, Artificial Sequence
  Chemically Synthetized
gagcgattagcgtgcgttc

SEQ ID NO: 71 - DNA, 21 bp, Artificial Sequence
  Chemically Synthetized
aaataacccgaacaaccgcga

SEQ ID NO: 72 - DNA, 26 bp, Artificial Sequence
  Chemically Synthetized
ttgttcgtcgtagtaagtgttcgcgc

SEQ ID NO: 73 - DNA, 22 bp, Artificial Sequence
  Chemically Synthetized
agtttttagatttacgtcgggc

SEQ ID NO: 74 - DNA, 20 bp, Artificial Sequence
  Chemically Synthetized
gaacaactcgaaaccgaacg

SEQ ID NO: 75 - DNA, 24 bp, Artificial Sequence
  Chemically Synthetized
gcgagttttgcgttcgttttcgcg

SEQ ID NO: 76 - DNA, 25 bp, Artificial Sequence
  Chemically Synthetized
tggtgatggaggaggtttagtaagt

SEQ ID NO: 77 - DNA, 27 bp, Artificial Sequence
  Chemically Synthetized
aaccaataaaacctactcctcccttaa

SEQ ID NO: 78 - DNA, 30 bp, Artificial Sequence
  Chemically Synthetized
accaccacccaacacacaataacaaacaca